(19) United States
(12) Patent Application Publication
(10) Pub. No.: US 2021/0308255 A1
(43) Pub. Date: Oct. 7, 2021

(54) ZIKA VIRUS VACCINE

(71) Applicant: Sumagen Canada Inc., London (CA)

(72) Inventors: Chil-Yong KANG, London (CA); Gyoung Nyoun KIM, London (CA); Kunyu WU, London (CA); Manki SONG, Seoul (KR); Jung-ah CHOI, Seoul (KR); Sangkyun LEE, Toronto (CA)

(73) Assignee: Sumagen Canada Inc., London (CA)

(21) Appl. No.: 17/220,040

(22) Filed: Apr. 1, 2021

Related U.S. Application Data
(60) Provisional application No. 63/003,436, filed on Apr. 1, 2020.

(51) Int. Cl. A61K 39/205; A61P 31/14; C12N 7/00

(52) U.S. Cl. CPC A61K 39/205; A61K 2039/575; C12N 7/00; A61P 31/14

(57) ABSTRACT

A recombinant vesicular stomatitits virus (rVSV) having a Zika virus (ZIKV) envelope (E) gene, a prime boost immunization combination against ZIKV including: (a) a prime vaccine or immunogenic composition comprising a recombinant vesicular stomatitis virus (rVSV) carrying a ZIKV envelope (E) protein, and (b) a boost vaccine or immunogenic composition comprising a rVSV carrying the same ZIKV E protein. The ZIKV gene can be genetically modified to encode a modified ZIKV E protein that elevates glycoprotein synthesis and trigger efficient humoral immune response.

### CROSS REFERENCE TO RELATED APPLICATIONS

This application claims the benefit of provisional patent application Ser. No. 63/003,436, filed Apr. 1, 2020, the contents of which are hereby incorporated by reference in its entirety.

### REFERENCE TO SEQUENCE LISTING

This application includes an electronically submitted sequence listing in .txt format. The .txt file contains a sequence listing entitled “0195924_0006_ST25.txt” created on Mar. 29, 2021 and is 25,944 bytes in size. The sequence listing contained in this .txt file is part of the specification and is hereby incorporated by reference herein in its entirety.

### FIELD OF THE INVENTION

The present invention relates to a novel prime-boost vaccines or immunogenic compositions comprising two different serotypes of recombinant vesicular stomatitis viruses and to the use of the novel prime-boost vaccine in methods for prophylactic vaccination regimens against Zika virus.

### BACKGROUND OF THE INVENTION

Throughout this application, various references are cited in square brackets to describe more fully the state of the art to which this invention pertains. The disclosure of these references is hereby incorporated by reference into the present disclosure.

Zika virus (ZIKV) infection causes a wide range of diseases. The most serious consequences of ZIKV infection are microcephaly and other congenital malformations of the fetus when women get the infection during pregnancy [1, 2]. In most cases, ZIKV infection causes mild symptoms, but in rare cases, it increases the risk of neurological complications such as Guillain-Barré syndrome [3, 4]. ZIKV was first found in monkeys in the Zika forest of Uganda in 1947 [5] and later it revealed itself in humans in 1952 [6] and in 1954 [7]. More recently, ZIKV outbreaks occurred in Yap Island in 2007 [8] and another outbreak was discovered in French Polynesia in 2013 [9] followed by larger outbreaks in Brazil and other parts of the Americas in 2015 and 2016 [10,11]. These recent outbreaks triggered international attention and a call for ZIKV vaccine development. ZIKV infection peaked in the Spring of 2016, however, the number of confirmed cases appears to have decreased significantly in 2017 as well as subsequent years. Although the ZIKV epidemic has recently subsided, there is always a possibility that it may re-emerge in different parts of the world.

ZIKV belongs to the Flaviviridae and is primarily transmitted by the Aedes aegypti mosquitos [12, 13]. The virus also can be transmitted by an infected person to their sexual partners [14]. ZIKV is an enveloped virus containing a positive sense single-stranded, non-segmented RNA genome consists of approximately 10,000 nucleotides. The genome encodes three structural proteins [capsid (C, 122 amino acids), precursor to the membrane (PrM 178 amino acids), and envelope (E, 504 amino acids)] and five non-structural proteins [15]. Unlike some other flaviviruses, ZIKV replicates well in vitro enabling it to produce a conventional live-attenuated virus vaccine or killed whole-virus vaccine. The surface antigen, E protein, of the ZIKV is responsible for the receptor binding to initiate the virus infection. Thus, there is strong merit to design a vaccine with the ZIKV E protein gene using a safe and effective delivery vector. Accordingly, most ZIKV vaccine designs utilize the PreM plus E genes.

A number of ZIKV vaccines have been developed and clinical trials for a dozen vaccines have been conducted [16]. Many different vaccine strategies are being employed including a conventional live-attenuated virus and killed whole-virus vaccines. In addition, a variety of vaccine platforms using DNA, purified ZIKV protein, mRNA, recombinant vector virus and chimeric vaccine are also in the developmental phase [16]. There are advantages and disadvantages for each of these strategies. Two independent groups have developed DNA vaccines using the PrM+E gene of ZIKV in early stage of ZIKV vaccine development [17, 18]. These DNA vaccines induced neutralizing antibodies. However, whether or not these DNA vaccines can provide complete protection remained to be determined. Subsequently numerous investigators have developed ZIKV vaccine candidates using conventional vaccine strategies such as a killed whole-virus vaccine using a purified ZIKV [19] and a live, attenuated Zika virus vaccine [20, 21].

An ideal ZIKV vaccine should induce completely protective immune responses, must be safe, relatively easy to administrate, and efficient for manufacturing. There is room for an improved ZIKV vaccine to meet all the criteria for an ideal ZIKV vaccine.

The Applicant has developed vaccines and system comprising a combination of vaccines that elicits a response against ZIKV.

### SUMMARY OF THE INVENTION

In accordance with the present disclosure, a recombinant vesicular stomatitis virus (rVSV) carries a Zika virus (ZIKV) envelope (E) protein or that carries a modified ZIKV E protein.

In one embodiment, the present disclosure is a recombinant vesicular stomatitis virus (rVSV) carrying a gene that encodes a Zika virus (ZIKV) envelope (E) protein or that encodes for a modified ZIKV E protein.

In one embodiment of the rVSV of the present disclosure, the rVSV further carries a gene that encodes a precursor to the membrane (PrM) protein.

In one embodiment of the rVSV of the present disclosure, the rVSV further carries a gene that encodes a ZIKV membrane (M) gene.

In another embodiment of the rVSV of the present disclosure, the gene encodes for the modified ZIKV E protein, the modified ZIKV E.

In another embodiment of the rVSV of the present disclosure, the modified ZIKV E protein includes a VSV G protein transmembrane domain and cytoplasmic tail (Gtc) at the C-terminus of the ZIKV E protein.

In another embodiment of the rVSV of the present disclosure, the modified ZIKV E protein includes a glycoprotein signal peptide at the N-terminus of the ZIKV E protein and a VSV G protein transmembrane domain and cytoplasmic tail (Gtc) at the C-terminus of the ZIKV E protein.

In another embodiment of the rVSV of the present disclosure, the glycoprotein signal peptide is a honeybee melittin signal peptide.

In another embodiment of the rVSV of the present disclosure, the rVSV is of Indiana (VSV_{Ind}) serotype.

In another embodiment of the rVSV of the present disclosure, the rVSV_{Ind} carries a mutant matrix protein (M), and wherein the mutant M protein includes a GML mutation (rVSV_{Ind}-GML).

In another embodiment of the rVSV of the present disclosure, the rVSV is of New Jersey (VSV_{NJ}) serotype.

In another embodiment of the rVSV of the present disclosure, the rVSV_{NJ} carries a mutant matrix (M) protein, and wherein the mutant M protein includes a GMM mutation (rVSV_{NJ}-GMM) or a GMML mutation (rVSV_{NJ}-GMML).

In another embodiment, the present disclosure provides for a ZIKV vaccine or immunogenic composition comprising a rVSV according to any embodiment of the present disclosure.

In another embodiment of the present disclosure provides for a prime boost immunization combination against Zika virus (ZIKV) including: (a) a prime vaccine or immunogenic composition comprising a recombinant vesicular stomatitis virus (rVSV) carrying a gene that encodes for ZIKV envelope (E) protein or that encodes for a modified ZIKV E protein, and (b) a boost vaccine or immunogenic composition comprising a rVSV carrying the same gene.

In one embodiment of the prime boost immunization combination against ZIKV of the present disclosure, the rVSV of (a) and the rVSV of (b) further carry a gene that encodes for a precursor to the membrane (PrM) protein.

In another embodiment of the prime boost immunization combination against ZIKV of the present disclosure, the rVSV of (a) and the rVSV of (b) further carry a gene that encodes for a ZIKV membrane (M) protein.

In another embodiment of the prime boost immunization combination against ZIKV of the present disclosure, the gene of the rVSV of the prime vaccine or immunogenic composition and the gene of the rVSV of the boost vaccine or immunogenic composition encode for the modified ZIKV E protein, the modified ZIKV E protein having a glycoprotein signal peptide at the N-terminus of the ZIKV E protein and a VSV G protein transmembrane domain and cytoplasmic tail (Gtc) at the C-terminus of the ZIKV E protein. In one aspect, the glycoprotein signal peptide is a honeybee melittin signal peptide.

In another embodiment of the prime boost immunization combination against ZIKV of the present disclosure, the gene of the rVSV of the prime vaccine or immunogenic composition and the gene of the rVSV of the boost vaccine or immunogenic composition encode for the modified ZIKV E protein, the modified ZIKV E protein a VSV G protein transmembrane domain and cytoplasmic tail (Gtc) at the C-terminus of the ZIKV E protein.

In another embodiment of the prime boost immunization combination against ZIKV of the present disclosure, the rVSV of the prime vaccine or immunogenic composition and the rVSV of the boost vaccine or immunogenic composition are of Indiana serotype (rVSV_{Ind}), or the rVSV of the prime vaccine or immunogenic composition and the rVSV of the boost vaccine or immunogenic composition are of New Jersey serotype (rVSV_{NJ}).

In another embodiment of the prime boost immunization combination against ZIKV of the present disclosure, the rVSV of the prime vaccine or immunogenic composition is Indiana (VSV_{Ind}) and the rVSV of the boost vaccine or immunogenic composition is New Jersey (VSV_{NJ}).

In another embodiment of the prime boost immunization combination against ZIKV of the present disclosure, the rVSV of the prime vaccine or immunogenic composition and the rVSV of the boost vaccine or immunogenic composition carry a mutant matrix (M) protein, and wherein when the rVSV is rVSV_{Ind} the mutant M protein includes a GML mutation (rVSV_{Ind}-GML), and when the rVSV is rVSV New Jersey serotype the mutant M protein includes a GMM mutation (rVSV_{NJ}-GMM) or a GMML mutation (rVSV_{NJ}-GMML).

In another embodiment, the present disclosure provides for a method for inducing an immune response in a mammal against ZIKV, comprising: (a) administering to the mammal an effective amount of a prime vaccine or immunogenic composition including a rVSV carrying a gene that encodes for a ZIKV envelope (E) protein or that encodes for a modified ZIKV E protein, and (b) administering to the subject a booster vaccine or immunogenic composition comprising a rVSV carrying the same gene.

In one embodiment of the method for inducing an immune response in a mammal against ZIKV of the present disclosure, the rVSV of (a) and the rVSV of (b) further carry a gene that encodes for a precursor to the membrane (PrM) protein.

In one embodiment of the method for inducing an immune response in a mammal against ZIKV of the present disclosure, the rVSV of (a) and the rVSV of (b) further carry a gene that encodes for a ZIKV membrane (M) protein.

In one embodiment of the method for inducing an immune response in a mammal against ZIKV of the present disclosure, the rVSV of the prime vaccine or immunogenic composition and the rVSV of the boost vaccine or immunogenic composition include the gene that encodes for the modified ZIKV E protein, the modified ZIKV E protein having a glycoprotein signal peptide at the N-terminus of the ZIKV E protein and a VSV G protein transmembrane domain and cytoplasmic tail (Gtc) at the C-terminus of the ZIKV E protein.

In one embodiment of the method for inducing an immune response in a mammal against ZIKV of the present disclosure, the glycoprotein signal peptide is a honeybee melittin signal peptide.

In one embodiment of the method for inducing an immune response in a mammal against ZIKV of the present disclosure, the ZIKV E protein includes a VSV G protein transmembrane domain and cytoplasmic tail (Gtc) at its C-terminus.

In one embodiment of the method for inducing an immune response in a mammal against ZIKV of the present disclosure, the rVSV of the prime vaccine or immunogenic composition is Indiana (rVSV_{Ind}) and the rVSV of the boost vaccine or immunogenic composition is rVSV_{Ind}, or the rVSV of the prime vaccine or immunogenic composition is New Jersey (rVSV_{NJ}) and the rVSV of the boost vaccine or immunogenic composition is rVSV_{NJ}.

In one embodiment of the method for inducing an immune response in a mammal against ZIKV of the present disclosure, the rVSV of the prime vaccine or immunogenic composition is Indiana (rVSV_{Ind}) and the rVSV of the boost vaccine or immunogenic composition is New Jersey (rVSV_{NJ}).

In one embodiment of the method for inducing an immune response in a mammal against ZIKV of the present disclosure, the rVSV of the prime vaccine or immunogenic composition and the rVSV of the boost vaccine or immunogenic composition include a mutant matrix (M) protein gene.

In one embodiment of the method for inducing an immune response in a mammal against ZIKV of the present disclosure, the rVSV is of Indiana serotype and the mutant M protein includes a GML mutation (rVSV_{Ind}-GML), or the rVSV is of New Jersey serotype and the mutant M protein includes a GMM mutation (rVSV_{NJ}-GMM) or a GMML mutation (rVSV_{NJ}-GMML).

In one embodiment of the method for inducing an immune response in a mammal against ZIKV of the present disclosure, the immune response includes a humoral and a cellular immune response.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1. (SEQ ID NOS: 13 and 14) Generation of four different rVSV-ZIKV constructs expressing the Zika virus envelope (E) protein. ZIKV genes were inserted into pTV-VSV between G and L genes. pTV-ZIKV-PrM+E contains ZIKV PrM+E genes, pTV-ZIKV-ME contains ZIKV ME genes, pTV-ZIKV-msp-E contains ZIKV E gene with honeybee melittin signal peptides (msp; 21 amino acids in blue), and pTV-ZIKV-msp-E-Gct contains E protein with msp at the NH₂-terminus (20 amino acids in green) and VSV G protein transmembrane and cytoplasmic tail (Gtc in red) at the COOH-terminus (29 amino acids in brown). 1: Leader, t: Trailer, N: Nucleocapsid, P: Phosphoprotein, M: Matrix, G: Glycoprotein, L: Large protein (RNA dependent RNA polymerase), T7P: T7 RNA polymerase promoter, HDV59: 59 nucleotides of hepatitis delta virus ribozyme.

FIGS. 2A to 2D. ZIKV E protein expression in cells infected with rVSV_{Ind}-ZIKV and rVSV_{NJ}-ZIKV. FIG. 2A) Detection of ZIKV E protein in infected cell lysates. FIG. 2B) Detection of ZIKV E protein in the pelleted viral particles. BHK21 cells were infected with an MOI of 6 recombinant VSV_{Ind}-ZIKVs for 6 hours. rVSV_{Ind}-ZIKV Infected cells were harvested and lysed. Infected cell culture supernatants were collected, and viral particles were pelleted by ultra-centrifugation through 30% sucrose cushion. The samples of infected cell lysates and pelleted viral particles were prepared and analyzed by Western Blot using an anti-ZIKV E antibody 1:3000 dilution. The rVSV_{Ind}-GML served as the negative control. Lane 1: rVSV_{Ind}-GML-PrM+E, Lane 2: rVSV_{Ind}-GML-ME, Lane 3: rVSV_{Ind}-GML-msp-E, Lane 4: rVSV_{Ind}-GML-msp-E-Gtc, Lane 5: rVSV_{Ind}-GML. FIG. 2C) Detection of ZIKV E protein in infected cell lysates. FIG. 2D) Detection of ZIKV E protein in the pelleted viral particles. BHK21 cells were infected with recombinant VSV_{NJ}-ZIKVs at MOI of 6 for 6 hours. Infected cells were harvested and lysed. Infected cell culture supernatants were collected, and viral particles were pelleted by ultra-centrifugation through a 30% sucrose cushion. The samples of infected cell lysates and pelleted viral particles were prepared and analyzed by Western Blot using anti-ZIKV E antibody at 1:3000 dilution. The rVSV_{NJ}-GMM served as the negative control. Lane 1: rVSV_{NJ}-GMM-PrM+E, Lane 2: rVSV_{NJ}-GMM-ME, Lane 3: rVSV_{NJ}-GMM-msp-E, Lane 4: rVSV_{NJ}-GMM-msp-E-Gtc, Lane 5: rVSV_{NJ}-GMM.

FIGS. 3A to 3C. Immunogold staining with Anti-VSV G protein and Anti-ZIKV E protein. FIG. 3A) Detection of VSV G protein (blue arrow) on the recombinant VSV_{Ind}-GML-ZIKV-msp-E-Gtc viral particles. FIG. 3B) Detection of ZIKV E protein (red arrow) on the recombinant VSV_{Ind}-GML-ZIKV-msp-E-Gtc viral particles. FIG. 3C) Diagram of rVSV-ZIKV pseudovirion. The rVSV_{Ind}-GML-ZIKV-msp-E-Gtc viruses were analysed by immunogold staining with anti-VSV G antibody (Kerafast) and anti-ZIKV E antibody (Kerafast), and samples were checked under a Philips CM10 electron microscope.

FIGS. 4A to 4C. Humoral Immune responses with different doses of rVSV-ZIKV. FIG. 4A) Humoral immune responses by rVSV-ZIKV-PrM+E immunization. Five mice per each group were prime and boost immunized intramuscularly at two-week intervals with PBS, live ZIKV (2×10⁹ PFU), rVSV-Mock or eight different doses of rVSV-ZIKV-PrM+E (from 1×10⁵ PFU to 5×10⁸ PFU) respectively. rVSV_{Ind} serotype was used for prime-immunization, then rVSV_{NJ} serotype was used for boost-immunization. Sera were collected from each mouse at two weeks after each immunization (Day 13 and 27). The serum IgG titer against the ZIKV E protein was analyzed by ELISA. FIG. 4B) Humoral immune responses by rVSV-ZIKV-msp-E-Gtc immunization. Five mice per each group were prime and boost immunized intramuscularly with PBS, rVSV-Mock, or eight different doses of rVSV-ZIKV-msp-E-Gtc (from 1×10⁵ PFU to 5×10⁸ PFU) at two-week intervals. rVSV_{Ind} serotype was used for prime-immunization, then rVSV_{NJ} serotype was used for boost-immunization. Sera were collected from each mouse at two weeks after each immunization (Day 13 and 27). The serum IgG titer against the ZIKV E protein was analyzed by ELISA. FIG. 4C) Comparative immune responses of rVSV-ZIKV-PrM+E, rVSV-ZIKV-msp-E-Gtc, rVSV-Mock, and live ZIKV. The immunizations were carried out with 5×10⁷ PFU and 5×10⁸ PFU of rVSV-ZIKV-PrM+E, rVSV-ZIKV-msp-E-Gtc, 5×10⁸ PFU of rVSV-Mock or 2×10⁹ PFU of live ZIKV as described in FIG. 4A and FIG. 4B. Statistical significance was determined by One-Way ANOVA with Tukey's correction (*, p<0.05; **, p<0.01; ***p<0.001; ****p<0.0001; ns, not significant). The data was presented as means with error bars of standard deviation. The dotted line across represents background.

FIG. 5. Evaluation of neutralizing antibody titers against live ZIKV. Five mice per group were prime and boost immunized intramuscularly with 2×10⁹ PFU of live ZIKV, rVSV-Mock, rVSV-ZIKV-PrM+E (5×10⁷ PFU and 5×10⁸ PFU), or rVSV-ZIKV-msp-E-Gtc (5×10⁷ PFU and 5×10⁸ PFU) at two-week intervals. Sera were collected at two weeks after the boost-immunization (Day 27). Sera diluted in 1/20, 1/40, 1/80, 1/160, and 1/320 were tested for neutralizing activity against ZIKV-Fortaleza/2015 (Brazil) strain. Serially-diluted mouse sera were incubated with ZIKV-Fortaleza/2015 (Brazil), and the mixture was added to Vero cells. Three days after the infection, plaques were counted. The decrease in the number of plaques was represented as % neutralization. Statistical significance was determined by One-Way ANOVA with Tukey's correction (*, p<0.05; ns, not significant). The data was presented as means with error bars of standard deviation.

FIG. 6. T Cell Responses Against ZIKV E Protein. Five mice per group were prime and boost immunized with 2×10⁹ PFU of live ZIKV, rVSV-Mock, rVSV-ZIKV-PrM+E (1×10⁸ PFU and 5×10⁸ PFU), or rVSV-ZIKV-msp-E-Gtc (1×10⁸ PFU and 5×10⁸ PFU) intramuscularly. Two weeks after the boost-immunization, the splenocytes were prepared and were stimulated with HIV-gag peptide (AMQMLKETI) (SEQ ID NO: 15) (as a control peptide, purple column), CD8-specific (blue column), or CD4-specific (red column) peptide of ZIKV E protein [Zika-E646 (GRLITANPVITESTE) (SEQ ID NO: 16) and Zika-E294 (IGVENRDFV) (SEQ ID NO: 17)]. The number of IFN-7 secreting cells was counted using the mouse IFN-7 ELISPOT kit. Statistical significance was determined by One-Way ANOVA with Tukey's correction (*, p<0.05; **, p<0.05; ns, not significant). The data was presented as means with error bars of standard deviation.

FIGS. 7A to 7C. In vivo protection against Lethal ZIKV Challenge. B6(Cg)-Ifnar 1^{tm1.2Ees}/J (Ifnar^{-/-}) mice were prime and boost immunized with 5×10⁸ PFU of rVSV-Mock, rVSV-ZIKV-PrM+E, or rVSV-ZIKV-msp-E-Gtc respectively via intramuscular route at two-week intervals. Mice were challenged intraperitoneally with 1×10³ PFU of ZIKV-Fortaleza/2015 (Brazil) at two weeks after boost-immunization. The losses of body weight and survival were monitored daily for 14 days after the challenge. FIG. 7A) Survival rate of Ifnar^{-/-} mice immunized with rVSV-Mock, rVSV-ZIKV-PrM+E, rVSV-ZIKV-msp-E-Gtc after challenge with ZIKV-Fortaleza/2015 (Brazil). The survival rate in challenged groups was compared by Log-rank test. The p value was 0.0029. FIG. 7B) Loss of body weight of rVSV-Mock, rVSV-ZIKV-PrM+E, rVSV-ZIKV-msp-E-Gtc immunized Ifnar^{-/-} mice after ZIKV-Fortaleza/2015 (Brazil) challenge. Body weight is presented as means of percentage of initial body weight with error bars of standard deviation. FIG. 7C) Experimental scheme of in vivo protection study using Ifnar^{-/-} mice.

### DETAILED DESCRIPTION OF THE INVENTION

### 1. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The articles “a” and “an” are used herein to refer to one or more than one (i.e., to at least one) of the grammatical object of the article.

The terms “animal” and “subject” as used herein includes all members of the animal kingdom including mammals, preferably humans.

The term “effective amount” as used herein means an amount effective and at dosages and for periods of time necessary to achieve the desired result.

“rVSV” is used to refer to a recombinant vesicular stomatitis virus.

The term “Indiana”, and “IND” are used to refer to the VSV serotype Indiana (VSV_{Ind}). The term “New Jersey”, and “NJ” are used to refer to the VSV serotype New Jersey (VSV_{NJ}).

“M_{WT}” “M(WT)” are used to refer to VSV having a wild type M gene.

“G22E” is used to refer to an M_{WT} gene in VSV_{NJ} having a glycine changed to a glutamic acid at position 22.

“G21E” is used to refer to an M_{WT} gene in VSV_{Ind} having a glycine changed to a glutamic acid at position 21.

“L110A” is used to refer to an M_{WT} gene in VSV_{NJ} having a leucine changed to alanine at position 110.

“L111A” is used to refer to an M_{WT} gene in VSV_{Ind} having a leucine changed to alanine at position 111.

“L110F” is used to refer to an M_{WT} gene in VSV_{NJ} having a leucine changed to phenylalanine at position 110.

“L111F” is used to refer to an M_{WT} gene in VSV_{Ind} having a leucine changed to phenylalanine at position 111.

“M51R” is used to refer to an M_{WT} gene in the VSV_{Ind} having a methionine changed to an arginine at position 51.

“M48R+M51R” or “M48R/M51R” are used to refer to an M_{WT} gene in VSV_{NJ} having a methionine changed to an arginine at positions 48 and 51 respectively.

“rVSV_{Ind}(GML)” is used to refer to an M_{WT} gene in VSV_{Ind} having the combined mutation G21E, M51R and one of L111A or L111F.

“rVSV_{NJ}(GMM)” is used to refer to an M_{WT} gene in VSV_{NJ} having the combined mutation G22E, M48R/M51R.

“rVSV_{NJ}(GMML)” is used to refer to an M_{WT} gene in VSV_{NJ} having the combined mutation G22E, M48R/M51R and one of L110A or L110F.

Although in the case of rVSV_{Ind} either L111A or L111F, and in the case of rVSV_{NJ} either L110A or L110F can be used, for improved stability, alanine (A) is preferred over phenylalanine (F).

The term “protein” as used herein is defined as a chain of amino acid residues, usually having a defined sequence. As used herein the term protein is inclusive of the terms “peptides” and “proteins”. The terms also encompass an amino acid polymer that has been modified.

### 2. Overview

The present invention features recombinant vesicular stomatitis viruses (rVSV), immunization platforms, immunization regimens and medicaments useful for inducing an immune response in a subject and preventing Zika virus (ZIKV) infection in the subject.

The rVSVs of the present invention feature a ZIKV envelope (E) gene.

The ZIKV gene can be genetically modified to encode a modified ZIKV E protein that elevates glycoprotein synthesis and triggers efficient humoral immune response. In one embodiment, the ZTKV E gene is genetically modified to produce ZIKV E proteins having a glycoprotein signal peptide at its N-terminus. Any glycoprotein signal peptide that allows the E protein to be glycosylated and involved in intracellular trafficking can be used, for example the honeybee melittin signal peptide. For example, the E gene is genetically modified to produce E proteins having a honeybee melittin signal peptide (msp) at its N-terminus or to produce E proteins having the msp at its N-terminus, and the transmembrane domain and cytoplasmic tail of the VSV glycoprotein (Gtc) to form pseudotype VSVs that trigger efficient humoral immune responses against the ZTKV E protein.

In another embodiment, the rVSV of the present invention also carries a PrM gene or M gene.

The platforms, regimens and medicaments comprise: (a) one vaccine or immunogenic composition comprising a recombinant vesicular stomatitis virus (rVSV) carrying a gene that encodes for a ZIKV envelope (E) protein or for a modified ZTKV E protein, and (b) another vaccine or immunogenic composition comprising a rVSV carrying the same gene that encodes for the ZIKV E protein or the modified ZIKV E protein. In one aspect, the E protein has a msp attached to its N-terminus.

In another embodiment, the present invention is a ZIKV E protein having a msp attached at the N-terminus of the ZIKV E protein.

In another embodiment, the present invention is a ZIKV E protein having a msp attached at the N-terminus of the ZTKV E protein and a transmembrane domain and cytoplasmic tail of the VSV glycoprotein (Gtc) at the C-terminus of the ZIKV E protein.

In another embodiment, the present invention is a ZIKV E protein having a transmembrane domain and cytoplasmic tail of the VSV glycoprotein (Gtc) at the C-terminus of the ZIKV E protein.

In aspects, the genes of the rVSVs of the present disclosure (including the VSV genes, mutated VSV genes and the genes that encode for ZIKV E protein, ZIKV M protein and ZTKV PrM protein and modifications thereof), including the rVSVs included in the vaccines or immunogenic compositions of the present disclosure, are codon optimized for expression in a mammalian cell, including a human cell.

### 3. Vaccines or Immunogenic Compositions of the Invention

The present invention further features vaccines or immunogenic compositions comprising an rVSV as described above. The vaccine or immunogenic compositions of the invention are suitable for administration to subjects in a biologically compatible form in vivo. The expression “biologically compatible form suitable for administration in vivo” as used herein means a form of the substance to be administered in which any toxic effects are outweighed by the therapeutic effects. The substances maybe administered to any animal or subject, preferably humans. The vaccines of the present invention may be provided as a lyophilized preparation. The vaccines of the present invention may also be provided as a solution that can be frozen for transportation. Additionally, the vaccines may contain suitable preservatives such as human albumin, bovine albumin, sucrose, glycerol or may be formulated without preservatives. If appropriate (i.e., no damage to the VSV in the vaccine), the vaccines may also contain suitable diluents, adjuvants and/or carriers.

A ZIKV vaccine or immunogenic composition according to this disclosure comprises a rVSV of the present disclosure.

In another embodiment of the present disclosure provides for a prime boost immunization combination against Zika virus (ZTKV) including: (a) a prime vaccine or immunogenic composition comprising a recombinant vesicular stomatitis virus (rVSV) carrying a gene that encodes for ZTKV envelope (E) protein or that encodes for a modified ZTKV E protein, and (b) a boost vaccine or immunogenic composition comprising a rVSV carrying the same gene.

In embodiments, the rVSV of the prime vaccine or immunogenic composition and the rVSV of the boost vaccine or immunogenic composition also carry a ZIKV PrM gene and/or a ZIKV M gene.

The rVSV of the prime vaccine or immunological composition may be of the same or different serotype as the rVSV of the boost vaccine or immunological composition. For example, both the prime and boost vaccines or immunogenic compositions are rVSV_{Ind}; or both the prime and boost vaccines or immunogenic compositions are rVSV_{NJ}; or the rVSV of the prime vaccine or immunogenic composition is rVSV_{Ind} and the rVSV of the boost vaccine or immunogenic composition is rVSV_{NJ}; or the rVSV of the prime vaccine or immunogenic composition is rVSV_{NJ} and the rVSV of the boost vaccine or immunogenic composition is rVSV_{Ind}.

The dose of the vaccine may vary according to factors such as the disease state, age, sex, and weight of the individual, and the ability of antibody to elicit a desired response in the individual. Dosage regime may be adjusted to provide the optimum therapeutic response. The dose of the vaccine may also be varied to provide optimum preventative dose response depending upon the circumstances.

### 4. Methods of Use

The present invention also features methods of inducing an immune response in a subject against ZIKV and/or preventing or treating a ZIKV infection in a subject comprising administering to the subject an effective amount of a combination of a vaccine or immunogenic composition of the present invention.

The present invention provides for a method for inducing an immune response in a subject to a ZIKV characterized in that said method comprises the following steps: (a) administering to the subject an effective amount of a prime vaccine or immunogenic composition including a rVSV carrying a ZIKV envelope (E) gene that encodes for a ZIKV E protein or that encodes for a modified ZICV E protein, and (b) administering to the subject a boost vaccine or immunogenic composition comprising a rVSV carrying the same ZIKV E gene that encodes the same ZIKV E protein or modified ZIKV E protein.

The rVSV of the prime vaccine or immunogenic composition may be of the same or different serotype as the rVSV of the booster vaccine or immunogenic composition. For example, both the prime and boost vaccines or immunogenic compositions are rVSV_{Ind}; or both the prime and boost vaccines or immunogenic compositions are rVSV_{NJ}; or the rVSV of the prime vaccine or immunogenic composition is rVSV_{Ind} and the rVSV of the boost vaccine or immunogenic composition is rVSV_{NJ}; or the rVSV of the prime vaccine or immunogenic composition is rVSV_{NJ} and the rVSV of the boost vaccine or immunogenic composition is rVSV_{Ind}.

The ZIKV E gene, in one embodiment, includes one of the genetically modified ZIKV E genes described above.

In aspects of the invention the methods for inducing an immune response in a mammal to a ZIKV and the methods for preventing or treating an infection caused by ZIKV may further comprise step (c) administering to the subject an effective amount of the vaccine or immunogenic composition of either step (a) or step (b). Step (c) may be administered to the subject more than one time over the course of inducing an immune response, preventing or treating.

Advantages

Advantages of the recombinant VSV-based platform technology of the present invention are first, a highly efficient prime-boost vaccination can be achieved with two antigenically distinct serotypes of rVSV vectors, because the vector immunity against the priming Indiana serotype (VSV_{Ind}) will not neutralize the boosting New Jersey serotype (VSV_{NJ}) vector. Thus, VSV_{NJ} carrying the same gene of interest as rVSV_{Ind} will provide maximum boost effects. A highly efficient prime-boost vaccination can also be achieved with the same serotype of rVSV vectors (i.e., both the prime and boost are rVSV_{Ind} or both the prime and boost are rVSV_{NJ}), because the pseudotype VSVs will carry both VSV G protein and ZIKV E protein on the surface. This pseudotype VSV can use either VSV receptor or ZIKV receptor to infect cells. Second, our genetically modified VSV_{Ind} M gene mutant (rVSV_{Ind}-GML) and genetically modified VSV_{NJ} M gene mutant (rVSV_{NJ}-GMM or GMML mutation (rVSV_{NJ}-GMML) vectors are completely safe, attenuated temperature-sensitive mutants [22]. Third, rVSV_{Ind}-GML, rVSV_{NJ}-GMM and rVSV_{NJ}-GMML vectors carrying foreign genes replicate highly efficiently. Therefore, high titer rVSV-based vaccines are relatively easy to prepare. Fourth, rVSV_{Ind}-GML, rVSV_{NJ}-GMM and rVSV_{NJ}-GMML vectors can accommodate a large-size foreign gene with up to 6,000 nucleotides, without decreasing the virus titer [24], and finally both serotypes of VSV have a very wide host range including humans.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### Examples

The examples are described for the purposes of illustration and are not intended to limit the scope of the invention.

We have employed a genetically modified dual serotype of vesicular stomatitis virus (VSV) platform technology [22, 23] to develop a prime-boost vaccine against ZIKV.

We have constructed rVSV carrying a modified ZIKV envelope (E) protein gene using genetically modified attenuated strains of both VSV_{Ind} and VSV_{NJ} as a prime-boost rVSV-ZIKV vaccine. We found prime immunization with rVSV_{Ind} carrying the ZIKV E gene with honeybee melittin signal peptide (msp) at the N-terminus and VSV G protein transmembrane domain and cytoplasmic tail (Gtc) at the C-terminus of the E gene followed by boost immunization with rVSV_{NJ} carrying the same modified E gene induced strong protective immune responses.

Here we present the induction of protective immune responses upon prime-boost vaccination of antigenically distinct dual serotypes of recombinant VSV vectors carrying a genetically modified E protein gene of ZIKV.

Methods

1 Cells and Viruses

BHK21 cells obtained from the American Type Culture Collection (ATCC) were cultured in Dulbecco's Modified Eagle Medium (DMEM) containing 5% fetal bovine serum (FBS). The BHK cell line expressing T7 bacteriophage RNA polymerase, BSR T7/5 cells, was provided by Drs. Buchholz and Conzelmann (Federal Research Center for Virus Diseases of Animals, Germany) [25] and cultured in DMEM containing 5% FBS and 500 μg/ml Geneticin (Gibco). Recombinant VSV-ZIKVs recovered from reverse genetics were purified three times by plaque picking and amplified in BHK21 cells as we have described previously [22].

ZIKV strain Fortaleza 2015 (ZIKV-Fortaleza/2015, Brazil) [26] was kindly provided by the Walter Reed Army Institute of Research (WRAIR). Vero cells (ATCC, CCL-81) were used to propagate the ZIKV. ZIKV was infected to Vero cells at the multiplicity of infection (MOI) of 0.1˜ 1 and harvested after three to four days post infection. The culture medium containing viruses was centrifuged at 5,000 rpm at 4° C. for 30 minutes to remove cell debris. The supernatant containing viruses was aliquoted and stored at −80° C. Plaque assay was performed to confirm the virus titer.

2 Plasmids Construction

The full-length cDNA clone of VSV_{Ind}-GML and VSV_{NJ}-GMM was cloned into the transcription vector pTV and named as pTV-VSV_{Ind}-GML and pTV-VSV_{NJ}-GMM as described previously [22]. To clone the ZIKV genes into the VSV vectors, the cDNA of ZIKV strain H/PH/2013 (European virus archive, EVAG) was used as the template.

For PrME gene cloning; the full length PrME gene was amplified by PCR using primer PrME-F and PrME-R (Table 1). The PCR product was cloned into the pBKS vector first, and after confirming the sequences, the correct PrME gene was cut out from the pBKS and inserted into pTV-VSV_{Ind}-GML and pTV-VSV_{NJ}-GMM between G and L gene using PmeI and MluI restriction sites. For ME gene cloning; the precursor to the membrane (Pr) gene was not included, only the full-length ME gene was amplified by the PCR using primer ME-F and PrME-R (Table 1). The PCR product was clone into pBKS vector first, and after confirming the sequences, the correct ME gene was cut out from pBKS and inserted into pTV-VSV_{Ind}-GML and pTV-VSV_{NJ}-GMM between the G and L genes using the PmeI and MluI restriction sites. For E gene with honeybee melittin signal peptide (msp) cloning; the signal peptide of E gene was replaced with msp. The msp gene (63 bps) was amplified by PCR using primer E-F and Emsp-mega (Table 1), and then the msp and the PCR product was used as a forward primer and primer PrME-R together to amplify the E gene. The PCR product was cloned into pBKS vector, and after the sequence were confirmed, the correct E gene with msp gene was cut out from pBKS and inserted into pTV-VSV_{Ind}-GML and pTV-VSV_{NJ}-GMM between G and L gene using PmeI and MluI restriction sites. For E gene with honeybee melittin signal peptide (msp) and VSV_{Ind} G protein transmembrane domain and cytoplasmic tail (Gtc) cloning; the signal peptide of E gene was replaced with msp gene and the transmembrane domain of E gene was replaced with Gtc. First, the msp gene was amplified by PCR using primer E-F and Emsp-mega (Table 1); second, the VSV_{Ind} Gtc was amplified using EGtc mega and EGtc R (Table 1) and then the PCR product of msp and Gtc were used as primers to amplify the E gene. The PCR product was cloned into the pBKS vector, and after confirming the sequences, the correct E gene with msp and Gtc was cut out from the pBKS and inserted into the pTV-VSV_{Ind}-GML and pTV-VSV_{NJ}-GMM between the G and L genes using PmeI and MluI restriction sites.

4 Western Blot Analysis

To prepare infected cell lysates, BHK, cells were infected with recombinant VSV-ZIKVs at an MOI of 6. At 6 hours post-infection, infected cells were collected and lysed in 100 μl of lysis buffer. To prepare samples of pelleted recombinant VSV-ZIKV particles, BHK₂₁ cells were infected with recombinant VSV-ZIKVs at an MOI of 0.1. At 20 hours post-infection, cell culture supernatants were collected and ultra-centrifuged through a 30% sucrose cushion at 35,000 rpm (Beckman SW-41) at 4° C. for 1 hour. The virus pellet was resuspended in 100 μl lysis buffer and stored at −80° C.

The standardized samples were analyzed by SDS-PAGE and detected by using the ECL Western blotting analysis kit (GE Healthcare). To detect ZIKV E protein, the mouse monoclonal anti-Zika E protein antibody (BioFront) (1:3,000) was used.

5 Immune Electron Microscopy

BHK21 cells were infected with rVSV_{Ind}-GML-ZIKV-msp-E-Gtc at an MOI of 0.1. At 20 hours post-infection, cell culture supernatants were collected and fixed with 0.1% glutaraldehyde (Merck), and then ultra-centrifuged through a 30% sucrose cushion at 35,000 rpm (Beckman SW-41) at 4° C. for 1 hour. The fixed virus pellet was resuspended in 400 μl TNE buffer and stored at 4° C. The virus surface proteins were labelled with immune-gold conjugated antibodies and analyzed by the Philips CM10 electron microscopy. To detect the Zika virus E protein, the mouse monoclonal anti-Zika E protein antibody 3E10D1 (Kerafast) (1:10) and goat anti-mouse IgG 15 nm gold conjugated antibody (EMS) were used. To detect VSV G protein, anti-VSV-G antibody 8G5F11 (Kerafast) (1:10) and goat anti-mouse IgG 15 nm gold conjugated antibody (EMS) were used.

6 Mice

Six-week-old C57BL/6 mice were purchased from Koatech (Pyeongtaek, Korea). Five to seven-week-old B6(Cg)-Ifnar 1^{tm1.2Ees}/J (Ifnar^{-/-}, JAX stock #028288) mice were purchased from Jackson Laboratories (Bar Harbor, Me., USA). Prior to the start of each animal experiment, all mice had an acclimation period of one week. All procedures were approved by the Institutional Animal Care and Use Committee at the International Vaccine Institute (IACUC Approval #2018-009).

7. Immunization

In all animal experiments, priming immunization was performed with rVSV_{Ind} serotype. Then two weeks after priming, boosting immunization was performed with rVSV_{NJ} serotype. Two weeks after each immunization, blood was collected from the retro-orbital plexus, and serum was obtained by centrifugation. The sera were stored at −80° C. until further analysis. It should be understood that boosting immunization may be performed at any time after the priming, including, without limitations, days, or weeks after priming.

For dose determination test, C57BL/6 mice were immunized with rVSV-ZIKV-PrM+E and rVSV-ZIKV-msp-E-Gtc constructs with 8 different doses from 1×10⁵ PFU to 5×10⁸ PFU, 2×10⁹ PFU of live ZIKV, 1 μg of animal cell-expressed ZIKV E protein or 5×10⁸ PFU of rVSV-Mock and PBS as a negative control via intramuscular injection. For the immunogenicity test, C57BL/6 mice were immunized with 5×10⁷ PFU or 5×10⁸ PFU of rVSV-ZIKV-PrM+E, and rVSV-ZIKV-msp-E-Gtc constructs, 2×10⁹ PFU of live ZIKV for positive control or 5×10⁸ PFU of rVSV-Mock as negative control through intramuscular injection.

8 Live ZIKV Challenge Protection Experiment

For live ZIKV challenge protection experiment, B6(Cg)-Ifnar 1^{tm1.2Ees}/J (Ifnar^{-/-}) mice were immunized with 5×10⁸ PFU of rVSV-ZIKV-PrM+E, rVSV-ZIKV-msp-E-Gtc or rVSV-Mock.

Two weeks after boost immunization, the mice were challenged with 1×10³ PFU of live ZIKV (ZIKV-Fortaleza/2015, Brazil) by intraperitoneal route. Body weight loss and survival rate were monitored every day for 14 days.

9 ELISA

To determine the ZIKV E protein-specific antibody titer, 96-well plates (Thermo Fisher Scientific, Waltham, Mass., USA) were coated with 2 μg/ml of ZIKV E protein (Cat #MBS319787, MyBioSource, San Diego, Calif., USA) in a 50 mM NaHCO₃ buffer for 16 hours at 4° C. After blocking with the blocking buffer [1% BSA (Merck, Darmstadt, Germany) in PBS] for 1 hour at 37° C., serum samples from mice were added into the plate as a 5-fold serial dilution, starting with a 1:30 dilution in the blocking buffer and incubated at 37° C. for 1 hour. Then, 1:3000 diluted HRP-conjugated goat anti-mouse IgG (Southern Biotech, Birmingham, Ala., USA) was applied and incubated at 37° C. for 1 hour. After washing, peroxidase substrate (TMB) solution (Millipore, Billerica, Mass., USA) was added to the each well and incubated within 10 minutes at room temperature (RT). The color development was stopped by adding 0.5N HCl (Merck, Darmstadt, Germany) and the optical density (OD) values were measured at wavelength 450 nm using the ELISA reader (Molecular Devices, San Jose, Calif., USA). The antibody titer was expressed as a reciprocal log 2 titer of serum dilution showing OD value of 0.2.

10 Plaque Reduction Neutralization Assay

ZIKV-specific neutralizing activity of the sera was analyzed by using the plaque reduction neutralization test (PRNT). Heat-treated mice sera were serially two-fold diluted with Eagle's Minimum Essential Medium (EMEM; containing 10% FBS). The diluted sera were incubated with 100 PFUs of ZIKV (ZIKV-Fortaleza/2015, Brazil) at 37° C. for 1 hour. Virus-sera mixtures were added to the Vero cells and incubated at 37° C. for 30 min. After incubation, the supernatant was removed and replaced with an agar overlay media containing 1% (w/v) low-melting agarose in EMEM (containing 2% FBS). After 3 days, the plates were fixed with 4% (v/v) formaldehyde solution (Sigma, Darmstadt, Germany). Then, plates were stained with 1% crystal violet solution (Sigma, Darmstadt, Germany) at RT for 1 hour and rinsed with water. Plaque numbers were counted and the neutralizing antibodies titer was determined as the serum dilution that inhibited 50% of the tested virus inoculum.

11 ELISpot Assay

To measure T cell responses after vaccination, an IFN-7 ELISpot assay was carried out using the Mouse IFN-7 ELISPOT Kit (BD Biosciences, San Jose, Calif., USA) according to the manufacturer's instructions. Briefly, spleens were removed from each mouse at two weeks after boost immunization, and then cellular suspensions of splenocytes were obtained by passing spleens through a 70 μm cell strainer (BD Bioscience, San Jose, Calif., USA). Red blood cells (RBCs) were removed by hemolysis using ACK Lysing Buffer (Thermo Fisher Scientific, Waltham, Mass., USA). 2×10⁵ cells were added into pre-coated BD™ ELISPOT plates in presence or absence of stimuli for 16 hours at 37° C. in 5% CO₂ incubator. After stimulation, cells and peptide stimulants were discarded and each well was soaked in distilled water for 3-5 minutes. After washing three times with Washing Buffer (BD Bioscience, San Jose, Calif., USA), 100 μl of diluted detection antibody [Biotinylated anti-mouse IFN-7 (BD Bioscience, San Jose, Calif., USA)] in Assay Diluent (BD Biosciences, San Jose, Calif., USA) was added to each well and incubated for 2 hours at RT. After washing, 1:100 diluted streptavidin-HRP (BD Bioscience, San Jose, Calif., USA) in Assay Diluent was added to each well, and then plates were incubated within 1 hour at RT. The plates were washed with Wash Buffer four times, and then washed with PBS twice. After washing, 1:50 diluted AEC chromogen with AEC Substrate Buffer (BD Bioscience, San Jose, Calif., USA) was added to each well. The spot development was stopped by washing with distilled water in each well. Air-dried plates were used to count the number of spots. The spots were counted using an ELISPOT reader (Molecular devices, San Jose, Calif., USA)

12. Statistics

The data on graphs were presented as means with error bars of standard deviation (SD). Statistical differences between mice groups were determined by one-way analysis of variance (ANOVA) with Tukey's correction for multiple comparisons. GraphPad Prism 8.3 was used to do the statistical analysis. Probability levels<0.05 (*, p<0.05; **, p<0.01; ***p<0.001; ****p<0.0001) were indicative of statistical significance.

Results

1. Construction of VSV-ZIKV E Recombinant Viruses

Humoral immune response against the ZIKV envelope (E) protein is believed to induce the virus neutralizing and protective antibodies. Thus, the E protein has been regarded as the primary target for ZIKV vaccine development [16]. We have chosen four different forms of the ZIKV E protein gene in order to determine which E protein construct will induce the strongest ZIKV neutralizing antibodies. These include an E protein gene with either PrM or M gene or an E gene with honeybee melittin signal peptide (msp) in order to enhance the efficiency of glycosylation and intracellular processing of the E protein as shown previously with other viral proteins [27, 28]. In addition, we added the transmembrane domain and cytoplasmic tail of the VSV glycoprotein (Gtc) to produce VSV pseudovirion with the ZIKV E protein which may induce a strong immune response against the ZIKV E protein (FIG. 1).

We have employed two different serotypes of genetically modified avirulent VSV, Indiana serotype (VSV_{Ind}-GML) and New Jersey serotype (VSV_{NJ}-GMM) [22]. The priming vaccine vector should be antigenically distinct from the boosting vaccine vector in order to maximize boost effects. The VSV system provides an ideal prime-boost vaccine vectors since the antibody against the VSV_{Ind} G protein does not neutralize VSV_{NJ} and vice versa. We have inserted ZIKV PrM gene with E gene and E gene with msp and Gtc genes (FIG. 1). These rVSV_{Ind}-GML-ZIKV E genes and rVSV_{NJ}-GMM-ZIKV E genes have been recovered by the VSV reverse genetics system as previously described [22]. However, as described above, a highly efficient prime-boost vaccination can also be achieved with the same serotype of rVSV vectors (i.e., both the prime and boost are rVSV_{Ind} or both the prime and boost are rVSV_{NJ}).

2. Intracellular and Secreted ZIKV E Proteins

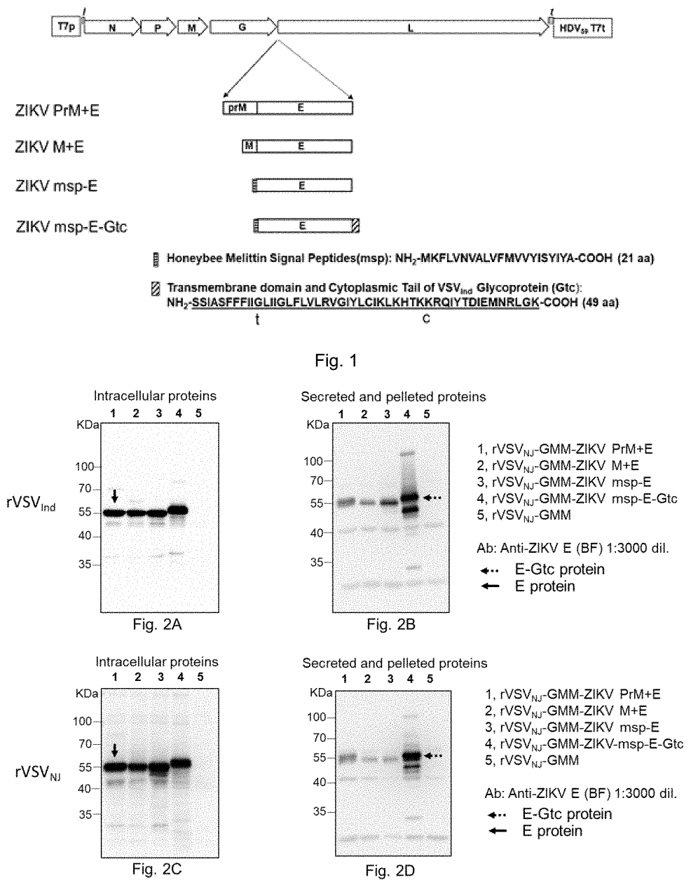

The ZIKV E protein gene expression was determined in the cells infected with rVSV_{Ind}-GML-ZIKV-E and rVSV_{NJ}-GMM-ZIKV-E viruses. BHK21 cells were infected with multiplicity of infection (MOI) of 6 and harvested 6 hours post infection and lysed cells for detection of the intracellular E proteins. The infected cell lysates were electrophoresed and the presence of E protein was determined by Western blot analysis using an anti-ZIKV E antibody. FIG. 2A shows the presence of E protein in all four lanes. Lanes 1, 2 and 3 representing rVSV_{Ind}-GML-ZIKV-PrM+E, rVSV_{Ind}-GML-ZIKV-M+E, and rVSV_{Ind}-GML-ZIKV-msp-E shows the same size E protein, but lane 4 representing rVSV_{Ind}-GML-ZIKV-msp-E-Gtc shows a slightly larger than 55 kDa E protein. These results show that the E protein expression levels from three different rVSV-ZIKVs are about the same and also indicates that PrM, M and msp are cleaved off after the expression. The Gtc at the C-terminus of the E protein makes the protein slightly larger (FIG. 2A). We have determined the rVSV particle associated extracellular E proteins by pelleting the rVSV-ZIKV-E recombinant virus infected BHK21 cell supernatant (FIG. 2B). The rVSV-ZIKV infected cell supernatant was pelleted by ultracentrifugation through a 30% sucrose cushion. The pelleted samples were analyzed by SDS-PAGE followed by Western blot using the anti-ZIKV E antibody. Lanes 1, 2, and 3 showed varying amounts of E protein (FIG. 2B). Most secreted ZIKV E proteins are associated with rVSV as a part of pseudotype particles. It is not clear why there are two different sizes of E proteins associated with the rVSV pseudotype at this point.

The same experiments have been carried out using the rVSV_{NJ}. GMM vector. We have constructed the same combinations of ZIKV E protein genes using rVSV_{NJ}-GMM (FIG. 2C and FIG. 2D). The smaller E protein in the pelletable sample is less with VSV_{NJ}-ZIKV-msp-E-Gtc.

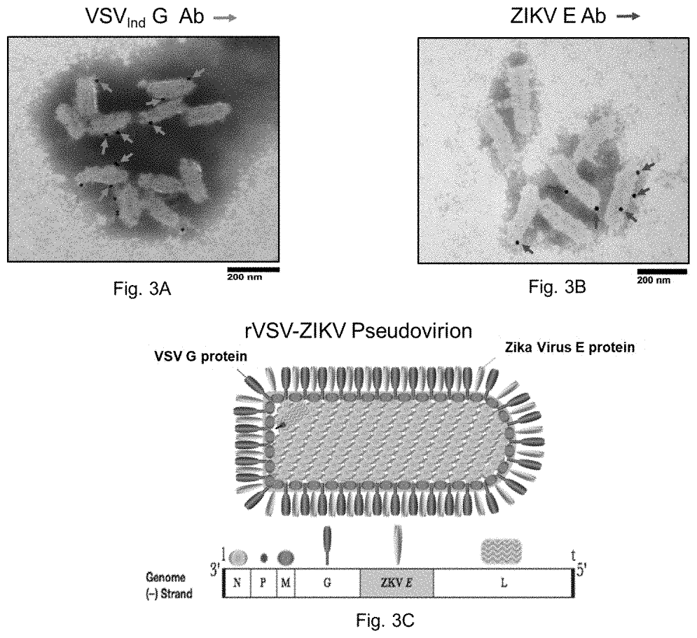

The results shown in FIG. 2 suggest strongly that rVSV pseudotypes are formed with the ZIKV E protein. To determine whether or not ZIKV E proteins are associated with rVSV virions as a pseudovirion, we have conducted an immune-electron microscopy using gold particle conjugated secondary antibodies. A partially purified rVSV_{Ind}-GML-ZIKV-msp-E-Gtc was incubated with either the anti-VSV G antibody or anti-ZIKV E antibody followed by a secondary anti-IgG conjugated with gold particles. FIG. 3A shows a detection of VSV G protein on the surface of rVSV_{Ind}-GML virions. FIG. 3B shows a detection of ZIKV E protein on the surface of rVSV_{Ind}-GML-ZIKV-msp-E-Gtc virions. FIG. 3C depicts a model for rVSV-ZIKV pseudovirion containing both VSV G protein and ZIKV E protein on the surface of the rVSV pseudovirions.

3 Immune Responses Induced by rVSV-ZIKV-PrM+E and rVSV-ZIKV-Msp-E-Gtc

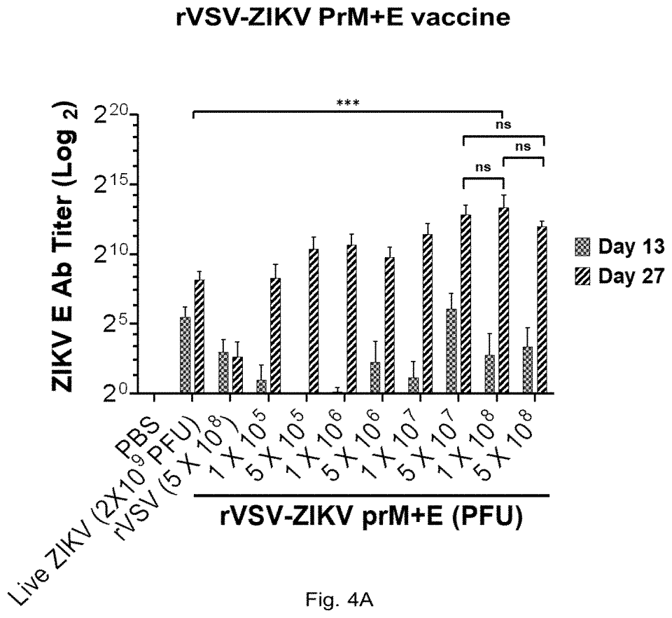

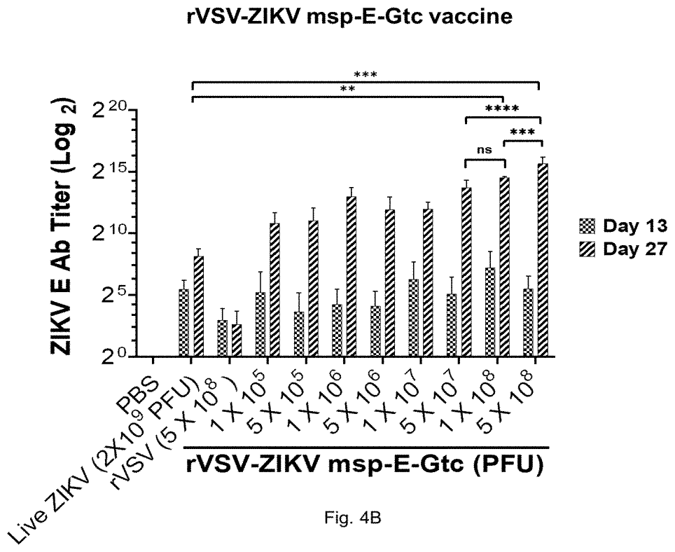

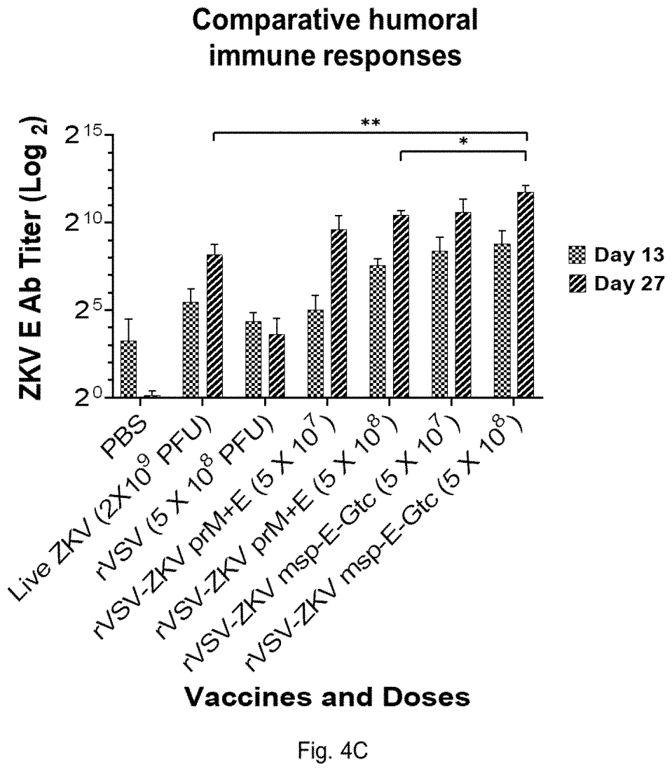

To determine optimal dose of rVSV-ZIKV vaccines, mice were immunized with eight different doses of rVSV-ZIKV-PrM+E, or rVSV-ZIKV-msp-E-Gtc. Mice were prime immunized with the rVSV_{Ind} serotype and boost immunized with rVSV_{NJ} serotype. Prime immunization with rVSV-ZIKV-PrM+E induced low level of ZIKV E specific antibodies at Day 13. Dosage of 5×10⁷ PFU/mouse or higher of rVSV-ZIKV-PrM+E induced slightly better ZIKV E specific antibody responses compare to the group of rVSV-mock, but it was not statistically significant (FIG. 4A). Priming immunization with rVSV_{Ind}-ZIKV-msp-E-Gtc induced E specific antibodies slightly better than the rVSV_{Ind}-ZIKV-prM+E in all dosage groups (FIG. 4B). After boosting immunization with rVSV_{NJ} serotype, over all ZIKV E specific antibody was generated in a significantly increased level in all dosage groups of rVSV-ZIKV-prM+E and rVSV-ZIKV-msp-E-Gtc (FIGS. 4A and 4B). When the E specific antibody responses were compared between groups immunized with rVSV-ZIKV-msp-E-Gtc and rVSV-ZIKV-prM+E vaccine groups, rVSV-ZIKV-msp-E-Gtc induced higher level of E specific antibodies compare to the mice immunized with rVSV-ZIKV-prM+E in the highest dosage group (5×10⁸ PFU/mouse, FIG. 4C). Mice immunized with rVSV_{Ind}-ZIKV-msp-E-Gtc induced equally strong or better immune responses in the highest dosage groups (5×10⁸ PFU/mouse) compare to mice immunized with a high dose of live ZIKV (FIG. 4C). Immune responses in mice immunized with a high dose of live ZIKV (2×10⁹ PFU) was strong as expected. The advantages for recombinant VSV-ZIKV vaccine are highly efficient immune responses, safety with the same protective immune responses, and the lower costs of vaccine production.

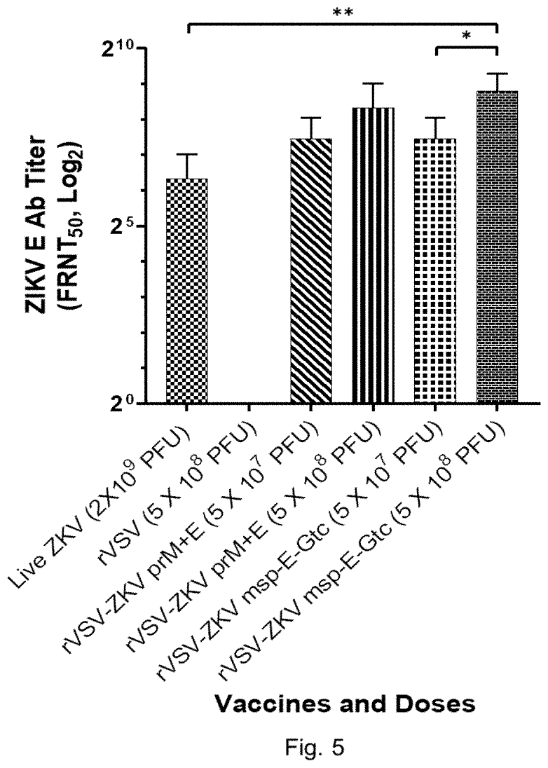

An effective vaccine should be able to induce strong neutralizing antibodies. For neutralization experiments, clinical isolate of ZIKV, ZIKV-Fortaleza/2015 (Brazil) [26], was pre-incubated with serially diluted antisera from immunized mice and residual infectious ZIKV was determined by plaque reduction neutralization assay on Vero cells and then the titer of neutralizing antibodies was determined. We found that immunization with both rVSV-ZIKV-prM+E and rVSV-ZIKV-msp-E-Gtc induced strong neutralizing antibodies. Prime-boost immunization with 5×10⁸ PFU of rVSV-ZIKV-msp-E-Gtc induced significantly higher neutralizing antibodies compared to prime-boost immunization of 2×10⁹ PFU of live ZIKV, the positive control group (FIG. 5). These immune response studies showed that rVSV_{Ind}-ZIKV-msp-E-Gtc priming followed by VSV_{NJ}-GMM-ZIKV-msp-E-Gtc boosting is a highly effective vaccination regimen to prevent ZIKV infection.

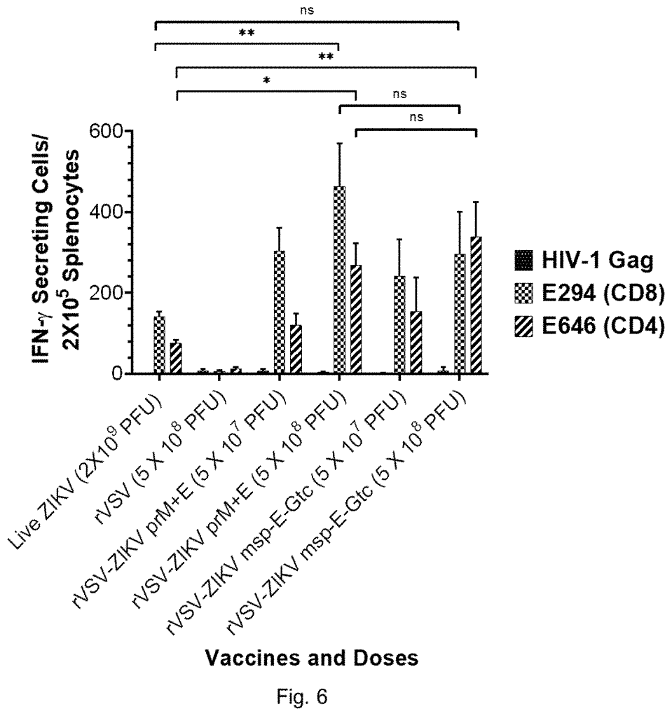

To further investigate immune responses of rVSV-ZIKV vaccine, we have tested the T cell immune responses against CD8 and CD4 specific peptides of ZIKV E protein by IFN-γ ELISpot assay. Splenocytes from both rVSV-ZIKV-PrM+E and rVSV-ZIKV-msp-E-Gtc immunized mice showed higher number of IFN-γ-secreting cells compare to mice immunized with live ZIKV, indicating that stronger T cell responses were induced by rVSV-ZIKV immunization. Interestingly, while rVSV-ZIKV-PrM+E immunized group showed higher T cell responses against CD8-specific peptide than rVSV-ZIKV-msp-E-Gtc immunized group, the rVSV-ZIKV-msp-E-Gtc immunized group showed similar or slightly higher T cell responses against CD4-specific peptide compare to the rVSV-ZIKV-PrM+E immunized group (FIG. 6). Because CD4+ T cells are well-known mediator for humoral immune responses, these results indicate that rVSV-ZIKV-msp-E-Gtc can induce stronger humoral responses, which is consistent with higher level of neutralizing antibody titers after rVSV-ZIKV-msp-E-Gtc immunization shown in FIG. 5.

4. Protection Against Lethal ZIKV Challenge in Ifnar^{-/-} Mice

The ultimate goal of any prophylactic vaccination is to protect the host upon challenging with lethal dose of the live virus. However, wild type mice are not suitable for ZIKV infection, since ZIKV-infected wild type mice do not show any evidence of morbidity or mortality. Thus, we have used the type 1 interferon receptor-deficient (B6(Cg)-Ifnar 1^{tm1.2Ees}/J, Ifnar^{-/-}) mice as an animal model for ZIKV challenge studies, since this Ifnar^{-/-} mouse model has been used for studies of pathogenesis of wide range of viruses [29, 30, 31], especially ZIKV [32, 33, 34]. Ifnar^{-/-} mice were immunized with 5×10⁸ PFU of rVSV_{Ind}-ZIKV-PrM+E or rVSV_{Ind}-ZIKV-msp-E-Gtc followed with boosting immunization with 5×10⁸ PFU of rVSV_{NJ}-ZIKV-PrM+E or rVSV_{NJ}-ZIKV-msp-E-Gtc in two-week intervals. In addition, we used rVSV_{Ind}-Mock and rVSV_{NJ}-Mock without the ZIKV gene insert as negative controls. We observed that all rVSV vectors with or without the ZIKV gene insert did not show any adverse effects to Ifnar^{-/-} mice prior to the ZIKV challenge. These results reconfirm that both serotypes of our rVSV vectors are safe to use as vaccine vectors.

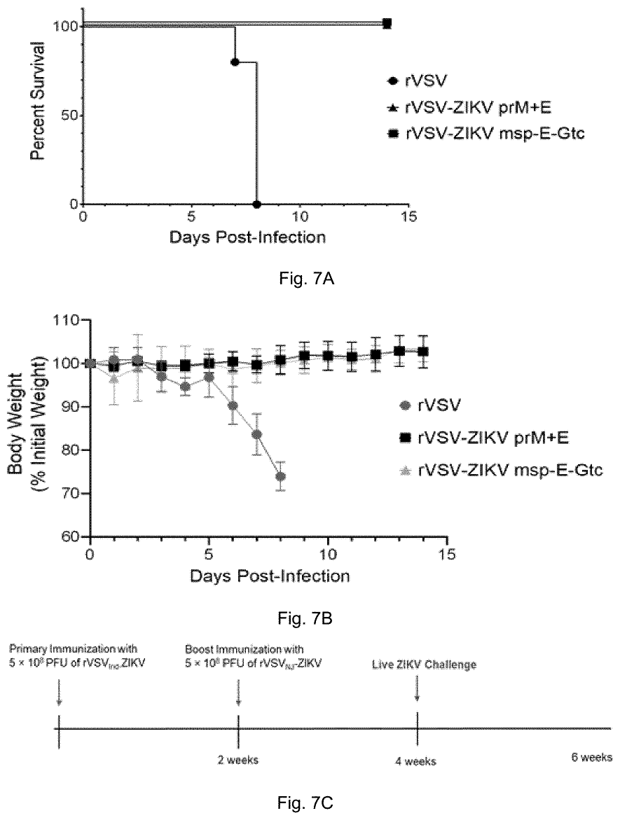

Two weeks after boosting immunization, 1×10³ PFU of ZIKV-Fortaleza/2015 (Brazil) was challenged to immunized Ifnar^{-/-} mice via intraperitoneal route, and their survival rate and loss of body weight were observed for 14 days post the virus challenge (PC). All rVSV-ZIKV-PrM+E and rVSV-ZIKV-msp-E-Gtc immunized mice survived after the ZIKV-Fortaleza/2015 (Brazil) challenge, while all rVSV-Mock vector-immunized negative control mice died within 8 days PC (FIG. 7A). In addition, loss of body weight was not observed in all rVSV-ZIKV-PrM+E and rVSV-ZIKV-msp-E-Gtc immunized mice, whereas rVSV-Mock vector immunized mice showed drastic loss of body weight (FIG. 7B). These results show clearly that both the rVSV-ZIKV-PrM+E vaccine and rVSV-ZIKV-msp-E-Gtc vaccine induced protective immune responses. Furthermore, the challenge studies clearly demonstrated that the modified E protein alone without PrM can induce protective immune responses against ZIKV infection.

TABLE 1
Primers used for cloning

| | SEQ | |
|---|---|---|
| | ID | |
| Primers | NO | Sequences |
| PrME-F | 18 | 5′-TCCCCGCGGGTTTAAACGATGAACGATATGAA |
| | | AAAAACTAACAGAATTCAAAATGGGCGCAGATACT |
| | | AGTGTCGGA-3′ |
| PrME-R | 19 | 5′-TCCCCGCGGACGCGTTTAAGCAGAG ACAGCT |
| | | GTGGAT-3′ |
| ME-F | 20 | 5′-TCCCCGCGGGTTTAAACGATGAACGATATGAA |
| | | AAAAACTAACAGAATTCAAAATGGTGGAACCAGAT |
| | | GACGTCGATT-3′ |
| E-F | 21 | 5′-TCCCCGCGGGTTTAAACGATGAACGATATGAA |
| | | AAAAACTAAC-3′ |
| Emsp-mega | 22 | 5′-GCTGACTCCTATGCACCTGATCGCATAGATGT |
| | | AAGAAATGTACAC-3′ |
| EGct-mega | 23 | 5′ATTTTTGGAGCAGCTTTCAAATCAAGCTCTATT |
| | | GCCTCTTTTTTCTTT-3′ |
| EGct-R | 24 | 5′-TCCCC GCGGACGCGTTTACTTCCCAAGTCGG |
| | | TTCATC-3′ |

NJ-M (F);
(SEQ ID NO: 25)
5′-tccccgcggttaattaagatgaacgatatgaaaaaaact-3′
NJ-M(M48R + M51R);
(SEQ ID NO: 26)
5′- tatcatataaatctcgatcctctcgtccgaagaagtca-3′
NJ-G (R);
(SEQ ID NO: 27)
5′-tccccgcggttaattaatttagcggaagtgagccat-3′
Ind(GLM) M (F);
SEQ ID NO: 28)
5′-cgggcggccgcttaattaaactatgaaaaaaagtaacagat -3′
Ind (GLM) M(M51R);
(SEQ ID NO: 29)
5′-catgagtgtctcgctcgtcaac-3′
Ind (GLM) M(L111A);
(SEQ ID NO: 30)
5′-aagatcttggcttttgcaggttcttc-3′
Ind(GLM) M (R);
(SEQ ID NO: 31)
5′-cgggcggccgctagactagctcatttg-3′

TABLE 2
Neucleotide Sequence Comparison between M Genes of VSV Indiana serotype,
Wild Type (SEQ ID NO: 1) and a Mutant G21E/L111A/M51R (SEQ ID NO 2)

| | |
|---|---|
| | 1 50 |
| SEQ ID NO: 1: | ATGAGTTCCT TAAAGAAGAT TCTCGGTCTG AAGGGGAAAG GTAAGAAATC |
| SEQ ID NO: 2: | ATGAGTTCCT TAAAGAAGAT TCTCGGTCTG AAGGGGAAAG GTAAGAAATC |
| | 51 100 |
| SEQ ID NO: 1: | TAAGAAATTA GGGATCGCAC CACCCCCTTA TGAAGAGGAC ACTAACATGG |
| SEQ ID NO: 2: | TAAGAAATTA GAAATCGCAC CACCCCCTTA TGAAGAGGAC ACTAACATGG |
| | 101 150 |
| SEQ ID NO: 1: | AGTATGCTCC GAGCGCTCCA ATTGACAAAT CCTATTTTGG AGTTGACGAG |
| SEQ ID NO: 2: | AGTATGCTCC GAGCGCTCCA ATTGACAAAT CCTATTTTGG AGTTGACGAG |
| | 151 200 |
| SEQ ID NO: 1: | ATGGACACTC ATGATCCGCA TCAATTAAGA TATGAGAAAT TCTTCTTTAC |
| SEQ ID NO: 2: | CGAGACACTC ATGATCCGCA TCAATTAAGA TATGAGAAAT TCTTCTTTAC |
| | 201 250 |
| SEQ ID NO: 1: | AGTGAAAATG ACGGTTAGAT CTAATCGTCC GTTCAGAACA TACTCAGATG |
| SEQ ID NO: 2: | AGTGAAAATG ACGGTTAGAT CTAATCGTCC GTTCAGAACA TACTCAGATG |
| | 251 300 |
| SEQ ID NO: 1: | TGGCAGCCGC TGTATCCCAT TGGGATCACA TGTACATCGG AATGGCAGGG |
| SEQ ID NO: 2: | TGGCAGCCGC TGTATCCCAT TGGGATCACA TGTACATCGG AATGGCAGGG |
| | 301 350 |
| SEQ ID NO: 1: | AAACGTCCCT TCTACAAGAT CTTGGCTTTT TTGGGTTCTT CTAATCTAAA |
| SEQ ID NO: 2: | AAACGTCCCT TCTACAAGAT CTTGGCTTTT GCAGGTTCTT CTAATCTAAA |
| | 351 400 |
| SEQ ID NO: 1: | GGCCACTCCA GCGGTATTGG CAGATCAAGG TCAACCAGAG TATCACGCTC |
| SEQ ID NO: 2: | GGCCACTCCA GCGGTATTGG CAGATCAAGG TCAACCAGAG TATCACGCTC |
| | 401 450 |
| SEQ ID NO: 1: | ACTGTGAAGG CAGGGCTTAT TTGCCACACA GAATGGGGAA GACCCCTCCC |
| SEQ ID NO: 2: | ACTGTGAAGG CAGGGCTTAT TTGCCACACA GAATGGGGAA GACCCCTCCC |
| | 451 500 |
| SEQ ID NO: 1: | ATGCTCAATG TACCAGAGCA CTTCAGAAGA CCATTCAATA TAGGTCTTTA |
| SEQ ID NO: 2: | ATGCTCAATG TACCAGAGCA CTTCAGAAGA CCATTCAATA TAGGTCTTTA |
| | 501 550 |
| SEQ ID NO: 1: | CAAGGGAACG GTTGAGCTCA CAATGACCAT CTACGATGAT GAGTCACTGG |
| SEQ ID NO: 2: | CAAGGGAACG GTTGAGCTCA CAATGACCAT CTACGATGAT GAGTCACTGG |
| | 551 600 |
| SEQ ID NO: 1: | AAGCAGCTCC TATGATCTGG GATCATTTCA ATTCTTCCAA ATTTTCTGAT |
| SEQ ID NO: 2: | AAGCAGCTCC TATGATCTGG GATCATTTCA ATTCTTCCAA ATTTTCTGAT |
| | 601 650 |
| SEQ ID NO: 1: | TTCAGAGATA AGGCCTTAAT GTTTGGCCTG ATTGTCGAGA AAAAGGCATC |
| SEQ ID NO: 2: | TTCAGAGATA AGGCCTTAAT GTTTGGCCTG ATTGTCGAGA AAAAGGCATC |
| | 651 700 |
| SEQ ID NO: 1: | TGGAGCTTGG GTCCTGGATT CTGTCAGCCA CTTCAAATGA |
| SEQ ID NO: 2: | TGGAGCTTGG GTCCTGGATT CTGTCAGCCA CTTCAAATGA |

TABLE 3
Amino Acid Sequence Comparison between M Proteins of VSV Indiana
serotype Wild Type (SEQ ID NO: 3) and a Mutant G21E/L111A/M151R (SEQ ID NO: 4)

| | |
|---|---|
| | 1 21 50 |
| SEQ ID NO: 3: | MSSLKKILGL KGKGKKSKKL GIAPPPYEED TNMEYAPSAP IDKSYFGVDE |
| SEQ ID NO: 4: | MSSLKKILGL KGKGKKSKKL EIAPPPYEED TNMEYAPSAP IDKSYFGVDE |
| | 51 100 |
| SEQ ID NO: 3: | MDTHDPHQLR YEKFFFTVKM TVRSNRPFRT YSDVAAAVSH WDHMYIGMAG |
| SEQ ID NO: 4: | RDTHDPHQLR YEKFFFTVKM TVRSNRPFRT YSDVAAAVSH WDHMYIGMAG |
| | 101 111 150 |
| SEQ ID NO: 3: | KRPFYKILAF LGSSNLKATP AVLADQGQPE YHAHCEGRAY LPHRMGKTPP |
| SEQ ID NO: 4: | KRPFYKILAF AGSSNLKATP AVLADQGQPE YHAHCEGRAY LPHRMGKTPP |
| | 151 200 |
| SEQ ID NO: 3: | MLNVPEHFRR PFNIGLYKGT VELTMTIYDD ESLEAAPMIW DHFNSSKFSD |
| SEQ ID NO: 4: | MLNVPEHFRR PFNIGLYKGT VELTMTIYDD ESLEAAPMIW DHFNSSKFSD |
| | 201 229 250 |
| SEQ ID NO: 3: | FRDKALMFGL IVEKKASGAW VLDSVSHFK |
| SEQ ID NO: 4: | FRDKALMFGL IVEKKASGAW VLDSVSHFK |

TABLE 4
Nucleotide Sequence Comparison between M Genes of VSV New Jersey
serotype Wild Type (SEQ ID NO: 5) and Mutants, G22E/M48R/M51R (SEQ ID NO: 6)
and G22E/L110A/M48R/M51R (SEQ ID NO: 7)

| | |
|---|---|
| | 1 50 |
| SEQ ID NO: 5: | ATGAGTTCCT TCAAAAAGAT TCTGGGATTT TCTTCAAAAA GTCACAAGAA |
| SEQ ID NO: 6: | ATGAGTTCCT TCAAAAAGAT TCTGGGATTT TCTTCAAAAA GTCACAAGAA |
| ID NO: 7: | ATGAGTTCCT TCAAAAAGAT TCTGGGATTT TCTTCAAAAA GTCACAAGAA |
| | 51 100 |
| SEQ ID NO: 5: | ATCAAAGAAA CTAGGCTTGC CACCTCCTTA TGAGGAATCA AGTCCTATGG |
| SEQ ID NO: 6: | ATCAAAGAAA CTAGAATTGC CACCTCCTTA TGAGGAATCA AGTCCTATGG |
| SEQ ID NO: 7: | ATCAAAGAAA CTAGAATTGC CACCTCCTTA TGAGGAATCA AGTCCTATGG |
| | 101 150 |
| SEQ ID NO: 5: | AGATTCAACC ATCTGCCCCA TTATCAAATG ACTTCTTCGG AATGGAGGAT |
| SEQ ID NO: 6: | AGATTCAACC ATCTGCCCCA TTATCAAATG ACTTCTTCGG ACGAGAGGAT |
| SEQ ID NO: 7: | AGATTCAACC ATCTGCCCCA TTATCAAATG ACTTCTTCGG ACGAGAGGAT |
| | 151 200 |
| SEQ ID NO: 5: | ATGGATTTAT ATGATAAGGA CTCCTTGAGA TATGAGAAGT TCCGCTTTAT |
| SEQ ID NO: 6: | CGAGATTTAT ATGATAAGGA CTCCTTGAGA TATGAGAAGT TCCGCTTTAT |
| SEQ ID NO: 7: | CGAGATTTAT ATGATAAGGA CTCCTTGAGA TATGAGAAGT TCCGCTTTAT |
| | 201 250 |
| SEQ ID NO: 5: | GTTGAAGATG ACTGTTAGAG CTAACAAGCC CTTCAGATCG TATGATGATG |
| SEQ ID NO: 6: | GTTGAAGATG ACTGTTAGAG CTAACAAGCC CTTCAGATCG TATGATGATG |
| SEQ ID NO: 7: | GTTGAAGATG ACTGTTAGAG CTAACAAGCC CTTCAGATCG TATGATGATG |
| | 251 300 |
| SEQ ID NO: 5: | TCACCGCAGC GGTATCACAA TGGGATAATT CATACATTGG AATGGTTGGA |
| SEQ ID NO: 6: | TCACCGCAGC GGTATCACAA TGGGATAATT CATACATTGG AATGGTTGGA |
| SEQ ID NO: 7: | TCACCGCAGC GGTATCACAA TGGGATAATT CATACATTGG AATGGTTGGA |
| | 301 350 |
| SEQ ID NO: 5: | AAGCGTCCTT TCTACAAGAT AATTGCTCTG ATTGGCTCCA GTCATCTGCA |
| SEQ ID NO: 6: | AAGCGTCCTT TCTACAAGAT AATTGCTCTG ATTGGCTCCA GTCATCTGCA |
| SEQ ID NO: 7: | AAGCGTCCTT TCTACAAGAT AATTGCTGCA ATTGGCTCCA GTCATCTGCA |
| | 351 400 |
| SEQ ID NO: 5: | AGCAACTCCA GCTGTGTTGG CAGACTTAAA TCAACCAGAG TATTATGCCA |
| SEQ ID NO: 6: | AGCAACTCCA GCTGTGTTGG CAGACTTAAA TCAACCAGAG TATTATGCCA |
| SEQ ID NO: 7: | AGCAACTCCA GCTGTGTTGG CAGACTTAAA TCAACCAGAG TATTATGCCA |
| | 401 450 |
| SEQ ID NO: 5: | CACTAACAGG TCGTTGTTTT CTTCCTCACC GACTCGGATT GATCCCACCG |
| SEQ ID NO: 6: | CACTAACAGG TCGTTGTTTT CTTCCTCACC GACTCGGATT GATCCCACCG |
| SEQ ID NO: 7: | CACTAACAGG TCGTTGTTTT CTTCCTCACC GACTCGGATT GATCCCACCG |
| | 451 500 |
| SEQ ID NO: 5: | ATGTTTAATG TGTCCGAAAC TTTCAGAAAA CCATTCAATA TTGGGATATA |
| SEQ ID NO: 6: | ATGTTTAATG TGTCCGAAAC TTTCAGAAAA CCATTCAATA TTGGGATATA |
| SEQ ID NO: 7: | ATGTTTAATG TGTCCGAAAC TTTCAGAAAA CCATTCAATA TTGGGATATA |
| | 501 550 |
| SEQ ID NO: 5: | CAAAGGGACT CTCGACTTCA CCTTTACAGT TTCAGATGAT GAGTCTAATG |
| SEQ ID NO: 6: | CAAAGGGACT CTCGACTTCA CCTTTACAGT TTCAGATGAT GAGTCTAATG |
| SEQ ID NO: 7: | CAAAGGGACT CTCGACTTCA CCTTTACAGT TTCAGATGAT GAGTCTAATG |
| | 551 600 |
| SEQ ID NO: 5: | AAAAAGTCCC TCATGTTTGG GAATACATGA ACCCAAAATA TCAATCTCAG |
| SEQ ID NO: 6: | AAAAAGTCCC TCATGTTTGG GAATACATGA ACCCAAAATA TCAATCTCAG |
| SEQ ID NO: 7: | AAAAAGTCCC TCATGTTTGG GAATACATGA ACCCAAAATA TCAATCTCAG |
| | 601 650 |
| SEQ ID NO: 5: | ATCCAAAAAG AAGGGCTTAA ATTCGGATTG ATTTTAAGCA AGAAAGCAAC |
| SEQ ID NO: 6: | ATCCAAAAAG AAGGGCTTAA ATTCGGATTG ATTTTAAGCA AGAAAGCAAC |
| SEQ ID NO: 7: | ATCCAAAAAG AAGGGCTTAA ATTCGGATTG ATTTTAAGCA AGAAAGCAAC |
| | 651 700 |
| SEQ ID NO: 5: | GGGAACTTGG GTGTTAGACC AATTGAGTCC GTTTAA |
| SEQ ID NO: 6: | GGGAACTTGG GTGTTAGACC AATTGAGTCC GTTTAA |
| SEQ ID NO: 7: | GGGAACTTGG GTGTTAGACC AATTGAGTCC GTTTAA |

TABLE 5
Amino Acid Sequence Comparison between M Proteins of VSV New Jersey
serotype Wild Type (SEQ ID NO: 8) and Mutants, G22E/M48R/M51R (SEQ ID NO: 9)
and G22E/L110A/M48R/M51R (SEQ ID NO: 10)

| | |
|---|---|
| | 1 22 48 50 |
| SEQ ID NO: 8: | MSSFKKILGF SSKSHKKSKK LGLPPPYEES SPMEIQPSAP LSNDFFGMED |
| SEQ ID NO: 9: | MSSFKKILGF SSKSHKKSKK LELPPPYEES SPMEIQPSAP LSNDFFGRED |
| SEQ ID NO: 10: | MSSFKKILGF SSKSHKKSKK LELPPPYEES SPMEIQPSAP LSNDFFGRED |
| | 51 100 |
| SEQ ID NO: 8: | MDLYDKDSLR YEKFRFMLKM TVRANKPFRS YDDVTAAVSQ WDNSYIGMVG |
| SEQ ID NO: 9: | RDLYDKDSLR YEKFRFMLKM TVRANKPFRS YDDVTAAVSQ WDNSYIGMVG |
| SEQ ID NO: 10: | RDLYDKDSLR YEKFRFMLKM TVRANKPFRS YDDVTAAVSQ WDNSYIGMVG |
| | 101 110 150 |
| SEQ ID NO: 8: | KRPFYKIIAL IGSSHLQATP AVLADLNQPE YYATLTGRCF LPHRLGLIPP |
| SEQ ID NO: 9: | KRPFYKIIAL IGSSHLQATP AVLADLNQPE YYATLTGRCF LPHRLGLIPP |
| SEQ ID NO: 10: | KRPFYKIIAA IGSSHLQATP AVLADLNQPE YYATLTGRCF LPHRLGLIPP |
| | 151 200 |
| SEQ ID NO: 8: | MFNVSETFRK PFNIGIYKGT LDFTFTVSDD ESNEKVPHVW EYMNPKYQSQ |
| SEQ ID NO: 9: | MFNVSETFRK PFNIGIYKGT LDFTFTVSDD ESNEKVPHVW EYMNPKYQSQ |
| SEQ ID NO: 10: | MFNVSETFRK PFNIGIYKGT LDFTFTVSDD ESNEKVPHVW EYMNPKYQSQ |
| | 201 250 |
| SEQ ID NO: 8: | IQKEGLKFGL ILSKKATGTW VLDQLSPFK |
| SEQ ID NO: 9: | IQKEGLKFGL ILSKKATGTW VLDQLSPFK |
| SEQ ID NO: 10: | IQKEGLKFGL ILSKKATGTW VLDQLSPFK |

Zika Virus Genes Inserted into VSV Vector:

Msp-E-Gtc
(SEQ ID NO: 11)
atgaaattcttagtcaacgttgcccttgtttttatggtcgtgtacatttc
ttacatctatgcgatcaggtgcataggagtcagcaatagggactttgtgg
aaggtatgtcaggtgggacttgggttgatgttgtcttggaacatggaggt
tgtgtcaccgtaatggcacaggacaaaccgactgtcgacatagagctggt
tacaacaacagtcagcaacatggcggaggtaagatcctactgctatgagg
catcaatatcggacatggcttcggacagccgctgcccaacacaaggtgaa
gcctaccttgacaagcaatcagacactcaatatgtctgcaaaagaacgtt
agtggacagaggctggggaaatggatgtggactttttggcaaagggagcc
tggtgacatgcgctaagtttgcatgctccaagaaaatgaccgggaagagc
atccagccagagaatctggagtaccggataatgctgtcagttcatggctc
ccagcacagtgggatgatcgttaatgacacaggacatgaaactgatgaga
atagagcgaaggttgagataacgcccaattcaccaagagccgaagccacc
ctggggggttttggaagcctaggacttgattgtgaaccgaggacaggcct
tgacttttcagatttgtattacttgactatgaataacaagcactggttgg
ttcacaaggagtggttccacgacattccattaccttggcacgctggggca
gacaccggaactccacactggaacaacaaagaagcactggtagagttcaa
ggacgcacatgccaaaaggcaaactgtcgtggttctagggagtcaagaag
gagcagttcacacggcccttgctggagctctggaggctgagatggatggt
gcaaagggaaggctgtectctggccacttgaaatgtcgcctgaaaatgga
taaacttagattgaagggcgtgtcatactccttgtgtaccgcagcgttca
cattcaccaagatcccggctgaaacactgcacgggacagtcacagtggag
gtacagtacgcagggacagatggaccttgcaaggttccagctcagatggc
ggtggacatgcaaactctgaccccagttgggaggttgataaccgctaacc
ccgtaatcactgaaagcactgagaactctaagatgatgctggaacttgat
ccaccatttggggactcttacattgtcataggagtcggggagaagaagat
cacccaccactggcacaggagtggcagcaccattggaaaagcatttgaag
ccactgtgagaggtgccaagagaatggcagtcttgggagacacagcctgg
gactttggatcagttggaggcgctctcaactcattgggcaagggcatcca
tcaaatttttggagcagctttcaaatcaagctctattgcctcttttttct
ttatcatagggttaatcattggactattcttggttctccgagttggtatt
tatctttgcattaaattaaagcacaccaagaaaagacagatttatacaga
catagagatgaaccgacttgggaagtaa
PreME
(SEQ ID NO: 12)
atgggcgcagatactagtgtcggaattgttggcctcctgctgaccacagc
tatggcagcggaggtcactagacgtgggagtgcatactatatgtacttgg
acagaaacgacgctggggaggccatatcttttccaaccacattggggatg
aataagtgttatatacagatcatggatcttggacacatgtgtgatgccac
catgagctatgaatgccctatgctggatgagggggtggaaccagatgacg
tcgattgttggtgcaacacgacgtcaacttgggttgtgtacggaacctgc
catcacaaaaaaggtgaagcacggagatctagaagagctgtgacgctccc
ctcccattccactaggaagctgcaaacgcggtcgcaaacctggttggaat
caagagaatacacaaagcacttgattagagtcgaaaattggatattcagg
aaccctggcttcgcgttagcagcagctgccatcgcttggcttttgggaag
ctcaacgagccaaaaagtcatatacttggtcatgatactgctgattgccc
cggcatacagcatcaggtgcataggagtcagcaatagggactttgtggaa
ggtatgtcaggtgggacttgggttgatgttgtcttggaacatggaggttg
tgtcaccgtaatggcacaggacaaaccgactgtcgacatagagctggtta
caacaacagtcagcaacatggcggaggtaagatcctactgctatgaggca
tcaatatcggacatggcttcggacagccgctgcccaacacaaggtgaagc
ctaccttgacaagcaatcagacactcaatatgtctgcaaaagaacgttag
tggacagaggctggggaaatggatgtggactttttggcaaagggagcctg
gtgacatgcgctaagtttgcatgctccaagaaaatgaccgggaagagcat
ccagccagagaatctggagtaccggataatgctgtcagttcatggctccc
agcacagtgggatgatcgttaatgacacaggacatgaaactgatgagaat
agagcgaaggttgagataacgcccaattcaccaagagccgaagccaccct
ggggggttttggaagcctaggacttgattgtgaaccgaggacaggccttg
acttttcagatttgtattacttgactatgaataacaagcactggttggtt
cacaaggagtggttccacgacattccattaccttggcacgctggggcaga
caccggaactccacactggaacaacaaagaagcactggtagagttcaagg
acgcacatgccaaaaggcaaactgtcgtggttctagggagtcaagaagga
gcagttcacacggcccttgctggagctctggaggctgagatggatggtgc
aaagggaaggctgtcctctggccacttgaaatgtcgcctgaaaatggata
aacttagattgaagggcgtgtcatactccttgtgtaccgcagcgttcaca
ttcaccaagatcccggctgaaacactgcacgggacagtcacagtggaggt
acagtacgcagggacagatggaccttgcaaggttccagctcagatggcgg
tggacatgcaaactctgaccccagttgggaggttgataaccgctaacccc
gtaatcactgaaagcactgagaactctaagatgatgctggaacttgatcc
accatttggggactcttacattgtcataggagtcggggagaagaagatca
cccaccactggcacaggagtggcagcaccattggaaaagcatttgaagcc
actgtgagaggtgccaagagaatggcagtcttgggagacacagcctggga
ctttggatcagttggaggcgctctcaactcattgggcaagggcatccatc
aaatttttggagcagctttcaaatcattgtttggaggaatgtcctggttc
tcacaaattctcattggaacgttgctgatgtggttgggtctgaacacaaa
gaatggatctatttcccttatgtgcttggccttagggggagtgttgatct
tcttatccacagctgtctctgcttaa

### REFERENCES

[1] Cauchemez S, Besnard M, Bompard P, Dub T, Guillemette-Artur P, Eyrolle-Guignot D, Salje H, DVan Kerkhove M, Abadie V, Garel C, Fontanet A, Mallet H-P. Association between Zika virus and microcephaly in French Polynesia, 2013-15: a retrospective study. Lancet 2016; 387 (10033): 2125-32 doi: 10.1016/50140-6736(16) 00651-6.
[2] Schuler-Faccini L, Ribeiro E M, Feitosa I M L, Horovitz D D G, Cavalcanti D P, Pessoa A, Doriqui M J R, Neri, J I, de Pina Neto J M, Wanderley H Y C, Cernach M, El-Husny A S, Pone M V S, Serao C L C, Sanseverino M T V, Brazilian Medical Genetics Society-Zika Embryopathy Task Force. Possible Association Between Zika Virus Infection and Microcephaly—Brazil, 2015. CDC MMWR Jan. 29, 2016/65(3); 59-62 https://www.cdc.gov/mmwr/volumes/65/wr/mm6503e2.htm.
[3] Brasil P, Sequeira P C, Freitas A D, Zogbi H E, Calvet G A, Valls de Souza R, Siqueira A M, Lima de Mendonca M C, Nogueira R M R, Bispo de Filippis A M, Solomon T. Guillain-Barré syndrome associated with Zika virus infection. Lancet 2016; 387:1482. doi: 10.1016/50140-6736(16)30058-7.
[4] Rozé B, Nqjioullqh F, Fergé J L, Dorléans F, Apetse K, Barnay J L, Daudens-Vaysse E, Brouste Y, Césaire R, Fagour L, Valentino R, Ledrans M, Mehdaoui H, Abel S, Leparc-Goffart I, Signate A, Cabié A. Guillain-Barré Syndrome Zika Working Group of Martinique. Guillain-Barré Syndrome Associated with Zika Virus Infection in Martinique in 2016: A Prospective Study. Clin Infect Dis 2017; 65(9):1462-1468. doi: 10.1093/cid/cix588.
[5] The Zika virus was discovered by Scottish virologist Alexander Haddow in 1947 and named after the Zika forest in Uganda where it was first found. The virus was isolated from a rhesus monkey.
[6] Dick G W A, Kitchen S F, Haddow A J. I. Isolations and serological specificity. Transactions of the Royal Society of Tropical Medicine and Hygiene 1952; 46(5): 509-520. doi: 10.1016/0035-9203(52)90042-4 ISSN 0035-9203.
[7] MacNamara FN. Zika virus: a report on three cases of human infection during an epidemic of jaundice in Nigeria. Trans R Soc Trop Med Hyg 1954; 48:139-45. 10.1016/0035-9203(54)90006-1.
[8] Duffy M R, Chen T-H, Hancock W T, Powers A M, Kool J L, Lanciotti R S, Pretrick M, Marfel M, Holzbauer S, Dubray C, Guillaumot L, Griggs, A, Bel M, Lambert A J, Laven J, Kosoy O, Panella A, Biggerstaff B J, Fischer M, Hayes E B. Zika Virus Outbreak on Yap Island, Federated States of Micronesia. New England Journal of Medicine 2009; 360 (24): 2536-2543. doi:10.1056/NEJMoa0805715. PMID 19516034.
[9] Cao Lormeau V M, Roche C, Teissier A, Robin E, Berry A L, Mallet H P, et al. Zika virus, French Polynesia, South Pacific, 2013. Emerg Infect Dis 2014; 20:1085-6. doi: 10.3201/eid2011.141380.
[10] Heukelbach J, Alencar C H, Kelvin A A, de Oliveira W K, Pamplona de Góes Cavalcanti L. Zika virus outbreak in Brazil. J Infect Dev Ctries 2016; 10(2):116-20. doi: 10.3855/jidc.8217.
[11] Hills S L, Fischer M, Petersen L R. Epidemiology of Zika Virus Infection. The Journal of Infect Dis 2017; 216, (suppl 10): S868-S874. doi.org/10.1093/infdis/jix434.
[12] Abushouk A I, Negida A, Ahmed H. An updated review of Zika virus. Journal of Clinical Virology 2016; 84: 53-58. doi:10.1016/j.jcv.2016.09.012. PMID 27721110.
[13] Ayres C F. Identification of Zika virus vectors and implications for control. The Lancet Infectious Diseases 2016; 16 (3): 278-9. doi:10.1016/S1473-3099(16)000736.
[14] Oster A M, Russell K, Stryker J E, Friedman A, Kachur R E, Petersen E E, et al. Update: Interim Guidance for Prevention of Sexual Transmission of Zika Virus—United States, 2016. MMWR. Morbidity and Mortality Weekly Report 2016; 65 (12): 323-5 doi:10.15585/mmwr.mm6512e3. PMID 27032078.
[15] Knipe D M, Howley P M (2007) Fields Virology 5^{th} Edition, Lippincott Williams & Wilkins).
[16] Barrett A D T. Current status of Zika vaccine development: Zika vaccines advance into clinical evaluation. NPJ Vaccines 2018; 3:24. doi: 10.1038/s41541-018-0061-9.
[17] Tebas P, Roberts C C, Muthumani K, et al. Safety and immunogenicity of an anti-Zika 50 virus DNA vaccine-preliminary report. N. Engl. J. Med 2017; 10.1056/NEJMoa1708120.
[18] Gaudinski M R, Houser K V, Morabito K M, et al. Safety, tolerability, and immunogenicity of two Zika virus DNA vaccine candidate in healthy adults: randomised, open-label, phase 1 clinical trials. Lancet 2017; 10.101016/50140-6736(17)33105-7.
[19] Modjarrad K, Lin L, George S L, et al. Preliminary aggregate safety and immunogenicity results from three trials of a purified inactivated Zika virus vaccine candidate: phase 1, randomized, double-blind, placebo-controlled clinical trials. Lancet 2017; 10.1016/S0140-6736(17)33106-9.
[20] Shan C, Muruato A E, Nunes B T D, et al. A live-attenuated Zika virus vaccine candidate induces sterilizing immunity in mouse models. Nat. Med 2017; 23:763-767. Doi:10.1038/nm4322.
[21] Shan C, Muruato A E, Jagger B W, et al. A single-dose live-attenuated vaccine prevents Zika virus pregnancy transmission and testis damage. Nat. Commun 2017; 8:676. doi10.1038/s41467-017-00737-8.
[22] Kim G N, Wu K, Hong, J, Awamleh Z & Kang C Y. Creation of matrix protein gene Variants of two serotypes of VSV as prime-boost vaccine vectors. J. Virol 2015; 89(12):6338-51 doi: 10.1128/JVI.00222-15.
[23] Kim G N, An H Y, Wu K, Banasikowska E, Harding M, Kang C Y. Matrix Protein Gene Variants of Two Serotypes of rVSV Make Effective Viral Vectors for Prime-Boost Vaccination. J. Human Virology & Retrovirology 2016; 4(1):00125 doi:10.15406/jhvrv.2016.04.00125.
[24] An, H Y, Kim, G N, Wu K and Kang, C Y. Genetically modified VSV_{NJ} vector is capable of accommodating a large foreign gene insert and allows high level gene expression, Virus Research 2013; 171:168-177.
[25] Buchholz U J, Finke S, Conzelmann K K. Generation of bovine respiratory syncytial virus (BRSV) from cDNA: BRSV NS2 is not essential for virus replication in tissue culture, and the human RSV leader region acts as a functional BRSV genome promoter. J Virol 1999; 73:251-9 PMCID: PMC113104.
[26] ZKV Strain Fortaleza 2015 (ZKV-Fortaleza/2015, Brazil) from Walter Reed Army Institute of Research, USA.
[27] Li Y, Luo L, Thomas D Y, Kang C Y. Control of glycosylation, intracellular localization and the secretion of HIV-1 gp120 by homologous and heterologous signal sequences. Virology 1994; 204, 266-278.
[28] Li Y, Bergeron J J, Luo L, Ou W J, Thomas D Y, and Kang C Y. Effects of inefficient cleavage of the signal sequence of HIV-1 gp120 on its association with calnexin, folding, and intracellular transport. Proc Natl Acad Sci USA 1996; 93:9606-9611.
[29] Rieger T, Merkler D, Gunther S. Infection of Type 1 Interferon Receptor-Deficient Mice with Various Old World Arenaviruses: A model for Studying Virulence and Host Species Barriers. PLoS ONE 2013; 8(8): e72290 https://doi.org/10.1371/journal.pone.0072290.
[30] Wei J, Ma Y, Wang L, Chi X, Yan R, Wang S, Li X, Chen X, Shao W, Chen J L. Alpha/beta interferon receptor deficiency in mice significantly enhances susceptibility of the animals to pseudorabies virus infection. Vet. Microbiol. 2017; 203:234-244. Doi: 10.1016/j.vetmic.2017.03.022. Epub 2017 Mar. 18.
[31] Wong G, Qiu X G. Type i interferon receptor knockout mice as models for infection of highly pathogenic viruses with outbreak potential. Zool Res 2018; 39:3-14. Doi: 10.24272/j.issn.2095-8137.2017.052.
[32] Bullard B L, Corder B N, Gorman M J, et al. Efficacy of a T Cell-Biased Adenovirus Vector as a Zika Virus Vaccine. Sci Rep 2018; 8(1):18017.
[33] Lopez-Camacho C, Abbink P, Larocca R A, et al. Rational Zika vaccine design via the modulation of antigen membrane anchors in chimpanzee adenoviral vectors. Nat Commun 2018; 9(1): 2441.
[34] Yockey L J, Jurado K A, Arora N, et al., Type I interferons instigate fetal demise after Zika virus infection. Sci Immunol 2018; 3(19).
[35] Vaccines and vaccination against yellow fever, WHO Position paper—June 2013 5 Jul. 2013, 88^{th} year, No. 27, 2013, 88, 269-284 http://www.who.int/wer
[36] Japanese Encephalitis Vaccines: WHO Position paper—February 2015 27 Feb. 2015, 90^{th} YEAR, No. 9, 2015, 90, 69-88 http://www.who.int/wer
[37] Tessier D C, Thomas D Y, Khouri H E, Laliberte F, and Vernet T. Enhanced secretion from insect cells of a foreign protein fused to the honeybee melittin signal peptide. Gene 1991; 98:177-83.
[38] Robinson C S, Whitt M A. The membrane-proximal stem region of vesicular stomatitis Virus G protein confers efficient virus assembly. J Virol 2000; 74:2239-46.
[39] Owens, R J, and Rose J K. Cytoplasmic domain requirement for incorporation of a foreign Envelope protein into vesicular stomatitis virus. J Virol 1993; 67:360-5.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. A recombinant vesicular stomatitis virus (rVSV) carrying a gene that encodes a Zika virus (ZIKV) envelope (E) protein or that encodes for a modified ZIKV E protein.

2. The rVSV of claim 1, wherein rVSV further carries a gene that encodes a precursor to the membrane (PrM) protein.

3. The rVSV of claim 1, wherein the rVSV further carries a gene that encodes a ZIKV membrane (M) gene.

4. The rVSV of claim 1, wherein the gene encodes for the modified ZIKV E protein, the modified ZIKV E.

5. The rVSV of claim 4, wherein the modified ZIKV E protein includes a VSV G protein transmembrane domain and cytoplasmic tail (Gtc) at the C-terminus of the ZIKV E protein.

6. The rVSV of claim 4, wherein the modified ZIKV E protein includes a glycoprotein signal peptide at the N-terminus of the ZIKV E protein and a VSV G protein transmembrane domain and cytoplasmic tail (Gtc) at the C-terminus of the ZIKV E protein.

7. The rVSV of claim 6, wherein the signal peptide is a honeybee melittin signal peptide.

8. The rVSV of claim 1, wherein the rVSV is of Indiana (VSV_{Ind}) serotype.

9. The rVSV of claim 8, wherein the rVSV_{Ind} carries a mutant matrix protein (M), and wherein the mutant M protein includes a GML mutation (rVSV_{Ind}-GML).

10. The rVSV of claim 1, wherein the rVSV is of New Jersey (VSV_{NJ}) serotype.

11. The rVSV of claim 10, wherein the rVSV_{NJ} carries a mutant matrix (M) protein, and wherein the mutant M protein includes a GMM mutation (rVSV_{NJ}-GMM) or a GMML mutation (rVSV_{NJ}-GMML).

12. A prime boost immunization combination against Zika virus (ZIKV) including: (a) a prime vaccine or immunogenic composition comprising a recombinant vesicular stomatitis virus (rVSV) carrying a gene that encodes for ZIKV envelope (E) protein or that encodes for a modified ZIKV E protein, and (b) a boost vaccine or immunogenic composition comprising a rVSV carrying the same gene.

13. The prime boost immunization combination against ZIKV of claim 12, wherein the rVSV of (a) and the rVSV of (b) further carry a gene that encodes for a precursor to the membrane (PrM) protein.

14. The prime boost immunization combination against ZIKV of claim 12, wherein the rVSV of (a) and the rVSV of (b) further carry a gene that encodes for a ZIKV membrane (M) protein.

15. The prime boost immunization combination against ZIKV of claim 12, wherein the gene of the rVSV of the prime vaccine or immunogenic composition and the gene of the rVSV of the boost vaccine or immunogenic composition encode for the modified ZIKV E protein, the modified ZIKV E protein having a glycoprotein signal peptide at the N-terminus of the ZIKV E protein and a VSV G protein transmembrane domain and cytoplasmic tail (Gtc) at the C-terminus of the ZIKV E protein.

16. The prime boost immunization combination against ZIKV of claim 15, wherein the glycoprotein signal peptide is a honeybee melittin signal peptide.

17. The prime boost immunization combination against ZIKV of claim 12, wherein the gene of the rVSV of the prime vaccine or immunogenic composition and the gene of the rVSV of the boost vaccine or immunogenic composition encode for the modified ZIKV E protein, the modified ZIKV protein having a VSV G protein transmembrane domain and cytoplasmic tail (Gtc) at the C-terminus of the ZIKV E protein.

18. The prime boost immunization combination against ZIKV of claim 12, wherein the rVSV of the prime vaccine or immunogenic composition and the rVSV of the boost vaccine or immunogenic composition are of Indiana serotype (rVSV_{Ind}), or the rVSV of the prime vaccine or immunogenic composition and the rVSV of the boost vaccine or immunogenic composition are of New Jersey serotype (rVSV_{NJ}).

19. The prime boost immunization combination against ZIKV of claim 12, wherein the rVSV of the prime vaccine or immunogenic composition is Indiana (VSV_{Ind}) and the rVSV of the boost vaccine or immunogenic composition is New Jersey (VSV_{NJ}).

20. The prime boost immunization combination against ZIKV of claim 12, wherein the rVSV of the prime vaccine or immunogenic composition and the rVSV of the boost vaccine or immunogenic composition carry a mutant matrix (M) protein, and wherein when the rVSV is rVSV_{Ind} the mutant M protein includes a GML mutation (rVSV_{Ind}-GML), and when the rVSV is rVSV New Jersey serotype the mutant M protein includes a GMM mutation (rVSV_{NJ}-GMM) or a GMML mutation (rVSV_{NJ}-GMML).

21. A method for inducing an immune response in a mammal against Zika virus (ZIKV), comprising: (a) administering to the mammal an effective amount of a prime vaccine or immunogenic composition including a rVSV carrying a gene that encodes for a ZIKV envelope (E) protein or that encodes for a modified ZIKV E protein, and (b) administering to the subject a booster vaccine or immunogenic composition comprising a rVSV carrying the same gene.

22. The method for inducing an immune response in a mammal against ZIKV of claim 21, wherein the rVSV of (a) and the rVSV of (b) further carry a gene that encodes for a precursor to the membrane (PrM) protein.

23. The method for inducing an immune response in a mammal against ZIKV of claim 21, wherein the rVSV of (a) and the rVSV of (b) further carry a gene that encodes for a ZIKV membrane (M) protein.

24. The method for inducing an immune response in a mammal against ZIKV of claim 21, wherein the rVSV of the prime vaccine or immunogenic composition and the rVSV of the boost vaccine or immunogenic composition include the gene that encodes for the modified ZIKV E protein, the modified ZIKV E protein having a glycoprotein signal peptide at the N-terminus of the ZIKV E protein and a VSV G protein transmembrane domain and cytoplasmic tail (Gtc) at the C-terminus of the ZIKV E protein.

25. The method for inducing an immune response in a mammal against ZIKV of claim 24, wherein the glycoprotein signal peptide is a honeybee melittin signal peptide.

26. The method for inducing an immune response in a mammal against ZIKV of claim 21, wherein the ZIKV E protein includes a VSV G protein transmembrane domain and cytoplasmic tail (Gtc) at its C-terminus.

27. The method of claim 21, wherein the rVSV of the prime vaccine or immunogenic composition is Indiana (rVSV_{Ind}) and the rVSV of the boost vaccine or immunogenic composition is rVSV_{Ind}, or the rVSV of the prime vaccine or immunogenic composition is New Jersey (rVSV_{NJ}) and the rVSV of the boost vaccine or immunogenic composition is rVSV_{NJ}.

28. The method of claim 21, wherein the rVSV of the prime vaccine or immunogenic composition is Indiana (rVSV_{Ind}) and the rVSV of the boost vaccine or immunogenic composition is New Jersey (rVSV_{NJ}).

29. The method of claim 21, wherein the rVSV of the prime vaccine or immunogenic composition and the rVSV of the boost vaccine or immunogenic composition include a mutant matrix (M) protein gene.

30. The method of claim 29, wherein the rVSV is of Indiana serotype and the mutant M protein includes a GML mutation (rVSV_{Ind}-GML), or the rVSV is of New Jersey serotype and the mutant M protein includes a GMM mutation (rVSV_{NJ}-GMM) or a GMML mutation (rVSV_{NJ}-GMML).

31. The method of claim 30, wherein the immune response includes a humoral and a cellular immune response.

32. A Zika virus (ZIKV) vaccine or immunogenic composition comprising the rVSV of claim 1.


SEQUENCE LISTING (31 sequences)

SEQ ID NO: 1 - DNA, 690 bp, Vesicular stomatitis virus
  misc_feature (1)..(690): M Gene of VSV Indiana serotype, Wild Type
atgagttccttaaagaagattctcggtctgaaggggaaaggtaagaaatctaagaaattagggatcgcaccacccccttatgaagaggacactaacatggagtatgctccgagcgctccaattgacaaatcctattttggagttgacgagatggacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttttgggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 2 - DNA, 690 bp, Artificial Sequence
  Synthetic M Gene of Mutant G21E/L111A/M51R
atgagttccttaaagaagattctcggtctgaaggggaaaggtaagaaatctaagaaattagaaatcgcaccacccccttatgaagaggacactaacatggagtatgctccgagcgctccaattgacaaatcctattttggagttgacgagcgagacactcatgatccgcatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaagatcttggcttttgcaggttcttctaatctaaaggccactccagcggtattggcagatcaaggtcaaccagagtatcacgctcactgtgaaggcagggcttatttgccacacagaatggggaagacccctcccatgctcaatgtaccagagcacttcagaagaccattcaatataggtctttacaagggaacggttgagctcacaatgaccatctacgatgatgagtcactggaagcagctcctatgatctgggatcatttcaattcttccaaattttctgatttcagagataaggccttaatgtttggcctgattgtcgagaaaaaggcatctggagcttgggtcctggattctgtcagccacttcaaatga

SEQ ID NO: 3 - protein, 229 aa, Vesicular stomatitis virus
  misc_feature (1)..(229): M Protein of VSV Indiana serotype Wild Type
MSSLKKILGLKGKGKKSKKLGIAPPPYEEDTNMEYAPSAPIDKSYFGVDEMDTHDPHQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 4 - protein, 229 aa, Artificial Sequence
  Synthetic M Protein of Mutant G21E/L111A/M51R
MSSLKKILGLKGKGKKSKKLEIAPPPYEEDTNMEYAPSAPIDKSYFGVDERDTHDPHQLRYEKFFFTVKMTVRSNRPFRTYSDVAAAVSHWDHMYIGMAGKRPFYKILAFAGSSNLKATPAVLADQGQPEYHAHCEGRAYLPHRMGKTPPMLNVPEHFRRPFNIGLYKGTVELTMTIYDDESLEAAPMIWDHFNSSKFSDFRDKALMFGLIVEKKASGAWVLDSVSHFK

SEQ ID NO: 5 - DNA, 686 bp, Vesicular stomatitis virus
  misc_feature (1)..(686): M Gene of VSV New Jersey serotype Wild Type
atgagttccttcaaaaagattctgggattttcttcaaaaagtcacaagaaatcaaagaaactaggcttgccacctccttatgaggaatcaagtcctatggagattcaaccatctgccccattatcaaatgacttcttcggaatggaggatatggatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 6 - DNA, 686 bp, Artificial Sequence
  Synthetic M Gene of Mutant G22E/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctctgattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 7 - DNA, 686 bp, Artificial Sequence
  Synthetic M Gene of Mutant G22E/L110A/M48R/M51R
atgagttccttcaaaaagattctgggattttcttcaaaaagtcacaagaaatcaaagaaactagaattgccacctccttatgaggaatcaagtcctatggagattcaaccatctgccccattatcaaatgacttcttcggacgagaggatcgagatttatatgataaggactccttgagatatgagaagttccgctttatgttgaagatgactgttagagctaacaagcccttcagatcgtatgatgatgtcaccgcagcggtatcacaatgggataattcatacattggaatggttggaaagcgtcctttctacaagataattgctgcaattggctccagtcatctgcaagcaactccagctgtgttggcagacttaaatcaaccagagtattatgccacactaacaggtcgttgttttcttcctcaccgactcggattgatcccaccgatgtttaatgtgtccgaaactttcagaaaaccattcaatattgggatatacaaagggactctcgacttcacctttacagtttcagatgatgagtctaatgaaaaagtccctcatgtttgggaatacatgaacccaaaatatcaatctcagatccaaaaagaagggcttaaattcggattgattttaagcaagaaagcaacgggaacttgggtgttagaccaattgagtccgtttaa

SEQ ID NO: 8 - protein, 229 aa, Vesicular stomatitis virus
  misc_feature (1)..(229): M Protein of VSV New Jersey serotype Wild Type
MSSFKKILGFSSKSHKKSKKLGLPPPYEESSPMEIQPSAPLSNDFFGMEDMDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 9 - protein, 229 aa, Artificial Sequence
  Synthetic M Protein of Mutant G22E/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIALIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 10 - protein, 229 aa, Artificial Sequence
  Synthetic M Protein of Mutant G22E/L110A/M48R/M51R
MSSFKKILGFSSKSHKKSKKLELPPPYEESSPMEIQPSAPLSNDFFGREDRDLYDKDSLRYEKFRFMLKMTVRANKPFRSYDDVTAAVSQWDNSYIGMVGKRPFYKIIAAIGSSHLQATPAVLADLNQPEYYATLTGRCFLPHRLGLIPPMFNVSETFRKPFNIGIYKGTLDFTFTVSDDESNEKVPHVWEYMNPKYQSQIQKEGLKFGLILSKKATGTWVLDQLSPFK

SEQ ID NO: 11 - DNA, 1578 bp, Artificial Sequence
  Synthetic Msp-E-Gtc
atgaaattcttagtcaacgttgcccttgtttttatggtcgtgtacatttcttacatctatgcgatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcaagctctattgcctcttttttctttatcatagggttaatcattggactattcttggttctccgagttggtatttatctttgcattaaattaaagcacaccaagaaaagacagatttatacagacatagagatgaaccgacttgggaagtaa

SEQ ID NO: 12 - DNA, 2076 bp, Artificial Sequence
  Synthetic PreME
atgggcgcagatactagtgtcggaattgttggcctcctgctgaccacagctatggcagcggaggtcactagacgtgggagtgcatactatatgtacttggacagaaacgacgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagctgtctctgcttaa

SEQ ID NO: 13 - protein, 21 aa, Apis mellifera
  misc_feature (1)..(21): Fig. 1 - Honeybee Melittin Signal Peptides(msp)
MKFLVNVALVFMVVYISYIYA

SEQ ID NO: 14 - protein, 49 aa, Vesicular stomatitis virus
  misc_feature (1)..(49): Fig. 1 - Transmembrane domain and Cytoplasmic Tail of VSVInd Glycoprotein (Gtc)
SSIASFFFIIGLIIGLFLVLRVGIYLCIKLKHTKKRQIYTDIEMNRLGK

SEQ ID NO: 15 - protein, 9 aa, Human immunodeficiency virus
  misc_feature (1)..(9): HIV-gag peptide
AMQMLKETI

SEQ ID NO: 16 - protein, 15 aa, Zika virus
  misc_feature (1)..(15): Zika-E646
GRLITANPVITESTE

SEQ ID NO: 17 - protein, 9 aa, Zika virus
  misc_feature (1)..(9): Zika-E294
IGVENRDFV

SEQ ID NO: 18 - DNA, 76 bp, Artificial Sequence
  Synthetic PrME-F
tccccgcgggtttaaacgatgaacgatatgaaaaaaactaacagaattcaaaatgggcgcagatactagtgtcgga

SEQ ID NO: 19 - DNA, 37 bp, Artificial Sequence
  Synthetic PrME-R
tccccgcggacgcgtttaagcagagacagctgtggat

SEQ ID NO: 20 - DNA, 77 bp, Artificial Sequence
  Synthetic ME-F
tccccgcgggtttaaacgatgaacgatatgaaaaaaactaacagaattcaaaatggtggaaccagatgacgtcgatt

SEQ ID NO: 21 - DNA, 42 bp, Artificial Sequence
  Synthetic E-F
tccccgcgggtttaaacgatgaacgatatgaaaaaaactaac

SEQ ID NO: 22 - DNA, 45 bp, Artificial Sequence
  Synthetic Emsp-mega
gctgactcctatgcacctgatcgcatagatgtaagaaatgtacac

SEQ ID NO: 23 - DNA, 48 bp, Artificial Sequence
  Synthetic EGct-mega
atttttggagcagctttcaaatcaagctctattgcctcttttttcttt

SEQ ID NO: 24 - DNA, 37 bp, Artificial Sequence
  Synthetic EGct-R
tccccgcggacgcgtttacttcccaagtcggttcatc

SEQ ID NO: 25 - DNA, 39 bp, Artificial Sequence
  Synthetic NJ-M (F)
tccccgcggttaattaagatgaacgatatgaaaaaaact

SEQ ID NO: 26 - DNA, 38 bp, Artificial Sequence
  Synthetic NJ-M(M48R+M51R)
tatcatataaatctcgatcctctcgtccgaagaagtca

SEQ ID NO: 27 - DNA, 36 bp, Artificial Sequence
  Synthetic NJ-G (R)
tccccgcggttaattaatttagcggaagtgagccat

SEQ ID NO: 28 - DNA, 41 bp, Artificial Sequence
  Synthetic Ind(GLM) M (F)
cgggcggccgcttaattaaactatgaaaaaaagtaacagat

SEQ ID NO: 29 - DNA, 22 bp, Artificial Sequence
  Synthetic Ind (GLM) M(M51R)
catgagtgtctcgctcgtcaac

SEQ ID NO: 30 - DNA, 26 bp, Artificial Sequence
  Synthetic Ind (GLM) M(L111A)
aagatcttggcttttgcaggttcttc

SEQ ID NO: 31 - DNA, 27 bp, Artificial Sequence
  Synthetic Ind(GLM) M (R)
cgggcggccgctagactagctcatttg